(19) United States
(12) Patent Application Publication
(10) Pub. No.: US 2017/0290929 A1
(43) Pub. Date: Oct. 12, 2017

(54) GRP78 TARGETED CONJUGATES

(71) Applicant: University of Iowa Research Foundation, Iowa City, IA (US)

(72) Inventors: Somya Kapoor, Iowa City, IA (US); Michael K. Schultz, Iowa City, IA (US)

(73) Assignee: University of Iowa Research Foundation, Iowa City, IA (US)

(21) Appl. No.: 15/316,585

(22) PCT Filed: May 14, 2015
(86) PCT No.: PCT/US2015/030856

§ 371 Date: Dec. 6, 2016

Related U.S. Application Data
(60) Provisional application No. 62/009,749, filed on Jun. 9, 2014.

(51) Int. Cl. A61K 49/00; A61K 31/16; A61N 5/00; A61K 51/08; A61K 33/24

(52) U.S. Cl. CPC A61K 49/0043; A61K 49/0056; A61K 51/088; A61K 33/24; A61N 5/00; A61K 31/16; A61K 47/48246; A61K 33/00

(57) ABSTRACT

Accordingly, certain embodiments of the invention provide a conjugate of formula (I): P-(X-D)n, wherein P is a peptide that binds to a glucose regulated protein 78 (GRP78); X is a direct bond or a linking group; D is a detectable agent; and n is 1 to 4. Certain embodiments of the invention provide a pharmaceutical composition comprising a conjugate of formula (I) and a pharmaceutically acceptable excipient. Certain embodiments of the invention provide a method for treating or preventing cancer in an animal (e.g., a human) comprising administering a therapeutically effective amount of a conjugate of formula (I) (e.g., a conjugate comprising a therapeutic radionuclide) to the animal.

### RELATED APPLICATION

This application claims the benefit of priority of U.S. Provisional Application Ser. No. 62/009,749 filed on Jun. 9, 2014, which application is herein incorporated by reference.

### GOVERNMENT FUNDING

This invention was made with government support under 1R01CA167632-01 and K25CA172218-01A1 awarded by the National Institutes of Health and NRC-HQ-12-G-38-0041 awarded by the Nuclear Regulatory Commission. The government has certain rights in the invention.

### BACKGROUND OF THE INVENTION

Glucose regulated protein 78 (GRP78) is a molecular chaperone that has long been considered to be confined to the endoplasmic reticulum, where it plays a major role in mediating appropriate protein folding and the unfolded protein response (UPR) in the cell (J. Li et. al. Cell Death and Differentiation (2008) 15, 1460-1471). Emerging evidences have demonstrated that GRP78 is exported from the internal organelle to the surface of the cell under conditions of stress (e.g., oxidative stress) (Amy. S. Lee Cancer Res (2007); 77:3496-3499). Although the mechanism and reasons for the protein to be present on the surface of cells is not well understood, it is increasingly being related to intracellular and extra cellular stresses that are associated with tumor microenvironment. The extent of GRP78 expression on cell surface has also been shown to be associated with the progression and stage of cancer (e.g., melanoma) (Liquing Zhuang et al. Histopathology (2009), 54, 462-470).

As the incidence of cancer is growing and therapies that provide lasting benefit have been slow to be developed, new targets for cancer diagnostics and therapy are needed. For example, metastatic melanoma has the fastest growing cancer incidence in the world today; however, current therapies increase life expectancy by only months. Additionally, drug resistance to traditional chemotherapeutics often arises quickly.

Thus, there is a need for agents that are useful for diagnosing, treating and/or preventing cancer.

### SUMMARY OF THE INVENTION

Accordingly, certain embodiments of the invention provide a conjugate of formula (I):

P-(X-D)ₙ (I)

wherein:
P is a peptide that binds to a glucose regulated protein 78 (GRP78);
X is a direct bond or a linking group;
D is a detectable agent; and
n is 1 to 4.

Certain embodiments of the invention provide a pharmaceutical composition comprising a conjugate of formula (I) and a pharmaceutically acceptable excipient.

Certain embodiments of the invention provide a method for treating or preventing cancer in an animal (e.g., a human) comprising administering a therapeutically effective amount of a conjugate of formula (I) (e.g., a conjugate comprising a therapeutic radionuclide) to the animal.

Certain embodiments of the invention provide a conjugate of formula (I) for use in medical therapy.

Certain embodiments of the invention provide a conjugate of formula (I) for the prophylactic or therapeutic treatment of cancer.

Certain embodiments of the invention provide the use of a conjugate of formula (I) to prepare a medicament for treating cancer in an animal (e.g. a human).

Certain embodiments of the invention provide a method of detecting a GRP78 molecule, comprising contacting a cell in vitro or in vivo with a conjugate of formula (I).

Certain embodiments of the invention provide a method of detecting cancer cells in a test tissue sample, comprising contacting the test sample with a conjugate of formula (I) and measuring a signal from the detectable agent, wherein a signal from the test sample that is greater than a signal from a non-cancerous control sample indicates the presence of cancer cells in the test tissue sample.

Certain embodiments of the invention provide a method of detecting cancer in an animal (e.g., a human), comprising administering a conjugate of any one of claims 1-48 to the animal and measuring a signal from the detectable agent, wherein a signal greater than a signal from a control animal without cancer indicates the animal has cancer.

Certain embodiments of the invention provide a method of determining the effectiveness of a cancer therapy in an animal (e.g., a human), comprising
1) administering a conjugate of formula (I) to the animal and measuring a first signal (e.g., a radioactive signal) from the detectable agent;
2) administering a cancer therapy;
3) administering a conjugate of formula (I) to the animal and measuring a second signal (e.g., a radioactive signal) from the detectable agent; and
4) comparing the first signal with the second signal, wherein the cancer therapy is effective if the second signal is less than the first signal.

Certain embodiments of the invention provide a kit comprising:
1) a conjugate of formula (I);
2) instructions for loading a radionuclide into the conjugate to generate a radiolabeled conjugate; and
4) instructions for administering the radiolabeled conjugate to an animal.

### BRIEF DESCRIPTION OF THE FIGURES

FIG. 1. PEP42 (CTVALPGGYVRKC) (SEQ ID NO:1) is a 13 amino acid cyclized peptide sequence which is believed to bind with GRP 78 with high specificity. FIG. 1A shows chemical structures of N-terminus conjugated DOTA and fluorescein conjugates of PEP42. FIG. 1B shows chemical structures of Lys-conjugated DOTA and fluorescein conjugates of PEP42. FIG. 1C is the pictorial representation of the PEP42 analogs.

FIG. 2. SK peptides. FIG. 2A shows the chemical structures of the synthesized peptides SK-1 (WDLAWMFRLPVG) (SEQ ID NO:2), SK-2 (WIFPWIQL) (SEQ ID NO:3), and SK-3 (GWAFSIPL) (SEQ ID NO:4). These peptides generally have hydrophobic amino acids at even positions, which are complimentary to the binding sites on the GRP78 according to the literature. FIG. 2B shows synthesized DOTA conjugates of peptides SK-1 (WDLAWMFRLPVG) (SEQ ID NO:2), SK-2 (WIFPWIQL) (SEQ ID NO:3), and SK-3 (GWAFSIPL) (SEQ ID NO:4). These peptides were labeled with Ga-68.

FIG. 3. Radiolabeling efficiency of PEP42 conjugates and Fluorescent Imaging for PEP42 conjugates. FIG. 3A. Shows the high radiolabeling efficiency of N-DOTA-PEP42. FIG. 3B. 10× confocal image of B16 tumor tissue cross section, which was incubated for 1 hour at room temperature with 20 μM of N-FAM PEP42.

FIG. 4. FIG. 4A. Pictorial representation of DOTA conjugated to the N-terminus of the SK-2 peptide. FIG. 4B. Counts per minute (cpm) v. Retention time (s) for ⁶⁸Ga-DOTA-SK2. FIG. 4C. ⁶⁸Ga-DOTA-SK2 binding curve (Counts per sec (cps) v. Concentration (nM)). 1-LN (10⁶) cells were incubated with increasing concentration of ⁶⁸Ga-DOTA-SK2 (0.01-100 nM) for an hour to obtain a saturation curve.

FIG. 5. Cell Surface Expression of GRP78. FIG. 5A shows the expression of GRP78 in B16 mouse melanoma tumor tissue cross section. The left figure shows the remarkable difference in the expression of GRP78 between the tumor tissue and surrounding muscle tissue by IHC using a goat-anti-GRP78 antibody. The right figure shows a high expression of GRP78 in B16 tumor tissue cross section by immunofluorescence. FIG. 5B. The left figure shows expression of GRP78 on the surface of 1-LN prostate cancer cells (Uma K. Misra et. al. The Journal of Biological Sciences (2002), Vol. 277, No. 44, 42082-42089) using a flow cytometer. The right figure shows the expression of GRP78 in 1-LN prostate cancer cells using microscopy. As melanoma cell lines show low expression in-vitro, 1-LN cells will be used for in-vitro peptide binding assays.

FIGS. 6A & B. Schematic of certain conjugates described herein. For example, the radionuclide may be altered depending on the use of the conjugate (e.g., ⁹⁰Y may be used for therapeutic purposes (A) and ⁶⁸Ga may be used for imaging (B)).

FIG. 7. GRP78 expression v. melanoma progression.

FIG. 8. Illustration of a conjugate of the invention binding to GRP78 at the surface of a cell.

FIG. 9. Synthesis of N-DOTA-PEP42.

FIG. 10. Illustration showing steps of a phage display study to select targeted peptides.

FIG. 11. PEP42 and phage binding assay.

FIG. 12. Illustration showing possible parameters for peptide design based on the GRP78 binding site.

FIG. 13. Cell surface expression of GRP78 and AKT/PI3K pro-survival pathway. Immunoprecipitation assay (left panel) and data (right panel).

FIGS. 14-19. Examples, using a model peptide, of conjugate/peptide modifications that may be made to alter, e.g., the affinity, selectivity and/or stability of a peptide/conjugate described herein. IC₅₀ is shown for each example.

FIG. 20. Stability of sample peptides in human serum.

FIG. 21. The left hand panel shows IHC staining on B-16 mouse melanoma tissue cross-section. There is a significantly high GRP78 expression in the tissue relative to the surrounding muscle tissue. The tumor tissue cross section was obtained from B-16 xenografts. The right hand panel shows a confocal image of the tumor tissue (20× magnification).

FIG. 22: (a) The increase in DHE oxidation shows increasing levels of ROS, which lead to increase in cell-surface GRP78 expression as shown in panels (b) and (c). Cell-surface GRP78 expression are analyzed by flow-cytometry using anti-human GRP78 alexa 488 tagged antibody. The cells are incubated at 4° C. for 40 min and fluorescence is measured and normalized with respect to the control.

### DETAILED DESCRIPTION

Glucose regulating peptide receptor (GRP78) is a protein that has long been considered to be confined to an intracellular organelle called the endoplasmic reticulum, where it plays a major role in appropriate protein folding and a cellular process known as the unfolded protein response. Emerging evidence is demonstrating that this protein can also be exported from the internal organelle to the surface of the cell. Although the mechanism and reasons for the protein to be present on the surface of cells is not well understood, it is increasingly being related to intra\extra cellular stresses that are associated with tumor mircoenvironment.

Accordingly, described herein are molecules (e.g., small amino acid sequences, i.e., peptides) that are designed to bind to cell surface GRP78. In certain embodiments, these molecules comprise a chelator that can be labeled with a radionuclide for diagnostic imaging (e.g., PET imaging) or radiation therapy for cancer. In certain embodiments, these molecules comprise a fluorescent molecular functional group that can be used for assays designed to identify and quantify the concentrate on of GRP78 on cell surfaces.

Thus, certain embodiments of the invention provide a conjugate of formula (I):

P-(X-D)ₙ (I)
wherein:
P is a peptide that binds to a glucose regulated protein 78 (GRP78);
X is a direct bond or a linking group;
D is a detectable agent or is a pharmaceutically active agent; and
n is 1 to 4.

Certain embodiments of the invention also provide a conjugate of formula (I):

P-(X-D)ₙ (I)
wherein:
P is a peptide that binds to a glucose regulated protein 78 (GRP78);
X is a direct bond or a linking group;
D is a detectable agent; and
n is 1 to 4.

Certain embodiments of the invention provide a conjugate of formula (I):

P-(X-D)ₙ (I)
wherein:
P is a peptide that binds to a glucose regulated protein 78 (GRP78);
X is a linking group;
D is a detectable agent; and
n is 1 to 4.

In certain embodiments, n is 1, 2, 3, 4. In certain embodiments, n is 1. In certain embodiments, n is 2. In certain embodiments, when n is greater than 1, -(X-D) is independently selected, and therefore, may be the same or different (e.g., one D may be a detectable agent and one D may be a pharmaceutically active agent; or, e.g., one D may be a radiolabeled chelating group and another D may be a fluorescent group).

The invention also provides processes and intermediates disclosed herein that are useful for preparing conjugates of formula I.

P: Peptides that Bind to GRP78

As described herein, P is a peptide that binds to a glucose regulated protein 78 (GRP78). Ideally, the peptide binds to GRP78 with a high affinity and specificity. Accordingly, in certain embodiments, the peptide binds to GRP78 with an IC₅₀ of about 100 pM to about 100 nM. In certain embodiments, the peptide has an IC₅₀ of about 300 pM to about 100 nM. In certain embodiments, the peptide has an IC₅₀ of about 500 pM to about 100 nM. In certain embodiments, the peptide has an IC₅₀ of about 800 pM to about 100 nM. In certain embodiments, the peptide has an IC₅₀ of about 1 nM to about 100 nM. In certain embodiments, the peptide has an IC₅₀ of about, e.g., 10 to about 90 nM, of about 20 to about 80 nM, of about 30 to about 70 nM, or of about 40 to about 60 nM.

In certain embodiments, a conjugate of formula (I) binds to GRP78 with an IC₅₀ of about 100 pM to about 100 nM. In certain embodiments, a conjugate of formula (I) has an IC₅₀ of about 300 pM to about 100 nM. In certain embodiments, a conjugate of formula (I) has an IC₅₀ of about 500 pM to about 100 nM. In certain embodiments, a conjugate of formula (I) has an IC₅₀ of about 800 pM to about 100 nM. In certain embodiments, a conjugate of formula (I) has an IC₅₀ of about 1 nM to about 100 nM. In certain embodiments, a conjugate of formula (I) has an IC₅₀ of about, e.g., 10 to about 90 nM, of about 20 to about 80 nM, of about 30 to about 70 nM, or of about 40 to about 60 nM.

In certain embodiments, the peptide is stable and/or has favorable pharmacodynamics (e.g., may be evaluated using imaging and bio distribution).

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, u-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆) alkyl, phenyl or benzyl ester or amide; or as an u-methylbenzyl amide). Other suitable amino and carboxy protecting groups are known to those skilled in the art (See for example, T. W. Greene, Protecting Groups In Organic Synthesis; Wiley: New York, 1981, and references cited therein). An amino acid can be linked to the remainder of a conjugate of formula I through the carboxy terminus, the amino terminus, or through any other convenient point of attachment, such as, for example, through the sulfur of a cysteine.

The term “peptide” describes a sequence of 2 to 25 (e.g., 3, 4, 5, 6, 7, 8, 9, 10, 11, 12, 13, 14, 15, 16, 17, 18, 19, 20, 21, 22, 23 or 24) amino acids (e.g. as defined hereinabove) or peptidyl residues. The sequence may be linear or cyclic. For example, a cyclic peptide can be prepared or may result from the formation of disulfide bridges between two cysteine residues in a sequence or may be cyclized by copper free click chemistry reactions. Peptide derivatives can be prepared as disclosed in U.S. Pat. Nos. 4,612,302; 4,853,371; and 4,684,620, or as described in the Examples herein below. Peptide sequences specifically recited herein are written with the amino terminus on the left and the carboxy terminus on the right.

A peptide can be linked to the remainder of a conjugate of formula I at its carboxy terminus, amino terminus, or through any other convenient point of attachment (e.g., attached to an amino acid located within the internal portion of the peptide), such as, for example, through the sulfur of a cysteine or through a free amine of an amino acid. Specifically, X may be covalently attached to the peptide at any synthetically feasible position. Ideally, X is covalently attached to the peptide at a position that does not interfere with or destroy its GRP78 binding capabilities and does not interfere with or destroy the functionality of the detectable agent. In certain embodiments, X is covalently attached through an amine group of an amino acid (e.g., generating an amide). In certain embodiments, X is covalently attached through an amine group of an amino acid, wherein the amino acid is not located at the N-terminus of the peptide. In certain embodiments, X is covalently attached through an alcohol group of an amino acid (e.g., generating an ester). In certain embodiments, X is covalently attached to a cysteine, tryptophan or glycine located at the N-terminus of the peptide through an amine group. In certain embodiments, X is covalently attached to an amine group of a lysine located within the internal portion of the amino acid sequence.

In certain embodiments the peptide is 4 to 20 amino acids in length. In certain embodiments, the peptide is 4 to 17 amino acids in length. In certain embodiments, the peptide is 5 to 16 amino acids in length. In certain embodiments, the peptide is 6 to 15 amino acids in length. In certain embodiments, the peptide is 7 to 14 amino acids in length. In certain embodiments, the peptide is 8 to 13 amino acids in length. In certain embodiments the peptide is 8 amino acids in length. In certain embodiments the peptide is 12 amino acids in length. In certain embodiments, the peptide is 13 amino acids in length.

Amino acids may be categorized based on their side chains. For example, alanine, isoleucine, leucine and valine have hydrophobic aliphatic side chains; phenylalanine, tryptophan and tyrosine have hydrophobic aromatic side chains; asparagine, methionine, cysteine, serine, glutamine and threonine have polar neutral side chains; aspartic acid and glutamic acid have charged acidic side chains; arginine, histidine and lysine have charged basic side chains; and glycine and proline are considered unique amino acids. Additionally, amino acids may be ranked based on a hydrophobicity index, which is a measure of the relative hydrophobicity, or how soluble an amino acid is in water. In a protein, hydrophobic amino acids are likely to be found in the interior, whereas hydrophilic amino acids are likely to be in contact with the aqueous environment. Based on this index, leucine, isoleucine, phenylalanine, tryptophan, valine, tyrosine, alanine, methionine and cysteine may be considered hydrophobic.

In certain embodiments, the peptide comprises at least one hydrophobic amino acid (e.g., leucine, isoleucine, phenylalanine, tryptophan, valine, tyrosine, alanine and cysteine). In certain embodiments, the peptide comprises at least two hydrophobic amino acids. In certain embodiments, the at least two hydrophobic amino acids may be the same. In certain embodiments, the at least two hydrophobic amino acids may be different. In certain embodiments, the peptide comprises at least three hydrophobic amino acids. In certain embodiments, the peptide comprises at least four hydrophobic amino acids. In certain embodiments, the peptide comprises at least five hydrophobic amino acids. In certain embodiments, the peptide comprises at least six hydrophobic amino acids. In certain embodiments, the peptide comprises at least seven hydrophobic amino acids. In certain embodiments, the hydrophobic amino acids may be the same. In certain embodiments, the hydrophobic amino acids may be different.

In certain embodiments, hydrophobic amino acids are located at the even positions within the peptide, which may result in increased binding affinity to GRP78. As used herein, the phrase “an even position” is used to refer to an amino acid at position 2, 4, 6, 8, 10, 12, 14, etc., when the amino acids within the peptide are numbered consecutively beginning with the first amino acid at the N-terminus of the peptide and moving towards the C-terminus.

Accordingly, in certain embodiments, the peptide comprises at least one even position hydrophobic amino acid. In certain embodiments, the peptide comprises at least two even position hydrophobic amino acids, independently selected. In certain embodiments, the peptide comprises at least three even position hydrophobic amino acids, independently selected. In certain embodiments, the peptide comprises at least four even position hydrophobic amino acids, independently selected.

In certain embodiments, the peptide comprises an amino acid sequence selected from formulas (II), (III), (IV), (V) and (VI):

X₁-A₁-A₁-A₂-A₂-A₂-A₃-A₃-A₃-A₄-A₂-A₅-A₅-A₁-X₁ (II)

X₁-A₀-A₇-A₀-A₂-A₀-A₁-A₀-A₅-A₀-A₃-A₀-A₃-X₁ (III)

X₁-A₀-A₂-A₀-A₃-A₀-A₂-A₀-A₂-X₁ (IV)

X₁-A₀-A₄-A₀-A₄-A₀-A₂-A₀-A₂-X₁ (V)

X₁-A₀-A₄-A₀-A₄-A₀-A₆-A₀-A₆-X₁ (VI)

wherein:

X₁ is 0-8 amino acids (e.g., any amino acid);

A₀ is any amino acid;

A₁ is independently an amino acid with a polar neutral side chain (e.g., asparagine, cysteine, glutamine, methionine, serine and threonine);

A₂ is independently an amino acid with a hydrophobic aliphatic side chain (e.g., alanine, isoleucine, leucine and valine);

A₃ is independently a unique amino acid (e.g., glycine and proline);

A₄ is independently an amino acid with a hydrophobic aromatic side chain (e.g., phenylalanine, tryptophan and tyrosine);

A₅ is independently an amino acid with a basic side chain (e.g., arginine, histidine and lysine);

A₆ is independently an amino acid with a hydrophobic aliphatic or aromatic side chain (e.g., alanine, isoleucine, leucine, valine, phenylalanine, tryptophan and tyrosine); and.

A₇ is independently an amino acid with an acidic side chain (e.g., aspartic acid and glutamic acid).

In certain embodiments, X₁ is 1, 2, 3, 4, 5, 6 or 7 amino acids (e.g., any amino acid).

In certain embodiments, the peptide comprises an amino acid sequence selected from CTVALPGGYVRKC (SEQ ID NO:1), WDLAWMFRLPVG (SEQ ID NO:2), WIFPWIQL (SEQ ID NO:3), and GWAFSIPL (SEQ ID NO:4).

In certain embodiments, the peptide comprises an amino acid sequence of formula (II).

In certain embodiments, the peptide consists of an amino acid sequence of formula (II).

In certain embodiments, the peptide comprises SEQ ID NO:1.

In certain embodiments, the peptide consists of SEQ ID NO:1.

In certain embodiments, the peptide comprises an amino acid sequence of formula (III).

In certain embodiments, the peptide comprises an amino acid sequence of formula (IIIa):

X₁-A₄-A₇-A₂-A₂-A₄-A₁-A₄-A₅-A₂-A₃-A₂-A₃-X₁ (IIIa).

In certain embodiments, the peptide consists of an amino acid sequence of formula (III).

In certain embodiments, the peptide consists of an amino acid sequence of formula (IIIa).

In certain embodiments, the peptide comprises SEQ ID NO:2.

In certain embodiments, the peptide consists of SEQ ID NO:2.

In certain embodiments, the peptide comprises an amino acid sequence of formula (IV).

In certain embodiments, the peptide comprises an amino acid sequence of formula (IVa):

X₁-A₄-A₂-A₄-A₃-A₄-A₂-A₁-A₂-X₁ (IVa).

In certain embodiments, the peptide consists of an amino acid sequence of formula (IV).

In certain embodiments, the peptide consists of an amino acid sequence of formula (IVa).

In certain embodiments, the peptide comprises SEQ ID NO:3.

In certain embodiments, the peptide consists of SEQ ID NO:3.

In certain embodiments, the peptide comprises an amino acid sequence of formula (V).

In certain embodiments, the peptide comprises an amino acid sequence of formula (Va):

X₁-A₃-A₄-A₂-A₄-A₁-A₂-A₃-A₂-X₁ (Va).

In certain embodiments, the peptide consists of an amino acid sequence of formula (V).

In certain embodiments, the peptide consists of an amino acid sequence of formula (Va).

In certain embodiments, the peptide comprises SEQ ID NO:4.

In certain embodiments, the peptide consists of SEQ ID NO:4.

In certain embodiments, the peptide comprises an amino acid sequence of formula (VI).

In certain embodiments, the peptide comprises an amino acid sequence of formula (VIa):

X₁-A₀-A₄ₐ-A₀-A₄ₐ-A₀-A₆ₐ-A₀-A₆ₐ-X₁ (VIa)

wherein:

A₄ₐ is independently selected from tryptophan and phenylalanine; and

A₆ₐ is independently selected from tryptophan, phenylalanine, isoleucine, leucine and tyrosine.

In certain embodiments, the peptide consists of an amino acid sequence of formula (VI).

In certain embodiments, the peptide consists of an amino acid sequence of formula (VIa):

X₁-A₀-A₄ₐ-A₀-A₄ₐ-A₀-A₆ₐ-A₀-A₆ₐ-X₁ (VIa)

wherein:

A₄ₐ is independently selected from tryptophan and phenylalanine; and

A₆ₐ is independently selected from tryptophan, phenylalanine, isoleucine, leucine and tyrosine.

### Linking Group X

In certain embodiments of the invention X is a linking group that joins the detectable agent (D) to a peptide that binds to a GRP78 (P). The nature of the linking group X is not critical provided the resulting conjugates retain the useful biological properties described herein (e.g., the peptide retains its GRP78 binding capabilities and the detectable agent retains its functionality).

In one embodiment of the invention the linking group has a molecular weight of from about 20 daltons to about 20,000 daltons.

In one embodiment of the invention the linking group has a molecular weight of from about 20 daltons to about 5,000 daltons.

In one embodiment of the invention the linking group has a molecular weight of from about 20 daltons to about 1,000 daltons.

In one embodiment of the invention the linking group has a molecular weight of from about 20 daltons to about 200 daltons.

In another embodiment of the invention the linking group has a length of about 5 angstroms to about 60 angstroms.

In another embodiment of the invention the linking group separates the peptide from the remainder of the conjugate of formula I by about 5 angstroms to about 40 angstroms, inclusive, in length.

In another embodiment of the invention the linking group is a divalent, branched or unbranched, saturated or unsaturated, hydrocarbon chain, having from 2 to 25 carbon atoms, wherein one or more (e.g. 1, 2, 3, or 4) of the carbon atoms is optionally replaced by (—O—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In another embodiment of the invention the linking group is a divalent, branched or unbranched, saturated or unsaturated, hydrocarbon chain, having from 2 to 10 carbon atoms, wherein one or more (e.g. 1, 2, 3, or 4) of the carbon atoms is optionally replaced by (—O—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In another embodiment of the invention the linking group is a divalent, branched or unbranched, saturated or unsaturated, hydrocarbon chain, having from 2 to 25 carbon atoms, wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In another embodiment of the invention the linking group is a divalent, branched or unbranched, saturated or unsaturated, hydrocarbon chain, having from 2 to 10 carbon atoms, wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In another embodiment of the invention the linking group is a divalent, branched or unbranched, saturated or unsaturated, hydrocarbon chain, having from 2 to 10 carbon atoms.

In another embodiment of the invention the linking group is a divalent, branched or unbranched, saturated hydrocarbon chain, having from 2 to 10 carbon atoms.

In another embodiment of the invention the linking group is a divalent, unbranched, saturated hydrocarbon chain, having from 2 to 10 carbon atoms.

In another embodiment of the invention the linking group is a divalent, unbranched, saturated hydrocarbon chain, having from 2 to 6 carbon atoms.

In another embodiment of the invention the linking group is a divalent, unbranched, saturated hydrocarbon chain, having from 2 to 4 carbon atoms.

In another embodiment of the invention the linking group comprises a polyethyleneoxy chain. In another embodiment of the invention the polyethyleneoxy chain comprises 2, 3, 4, 5, 6, 7, 8, 9, or 10 repeating ethyleneoxy units.

In another embodiment of the invention the linking group is —C(═O)—.

In another embodiment of the invention the linking group is a divalent radical formed from a protein.

In another embodiment of the invention the linking group is a divalent radical formed from a peptide.

In another embodiment of the invention the linking group is a divalent radical formed from an amino acid.

In another embodiment of the invention the carboxylic acid of the detectable group is reacted with an amine of the peptide to form an amide bond.

In certain embodiments of the invention, X is a direct bond. In certain embodiments, X is a direct bond and P is bonded to D through an amide bond.

### D: Detectable Agent or Pharmaceutically Active Agent

As described herein, conjugates of formula (I) comprise one or more detectable agents. Detectable agents include, but are not limited to, fluorescent groups and chelating groups, which may be labeled with radionuclides. When more than one detectable group is attached to the peptide, the detectable groups and linking groups are independently selected, and therefore, may be the same or may be different. For example, in certain embodiments, a chelating group, which may be labeled with a radionuclide, and a fluorescent group may be linked to a peptide via individual linking groups (X).

Accordingly, in certain embodiments, the detectable agent (D) comprises a chelating group, which may be labeled with a radionuclide. Thus, depending on the type of radionuclide selected, the conjugates may be used for, e.g., diagnostic imaging (e.g., PET imaging, MRI) or radiation therapy for cancer (e.g., which may circumvent drug resistance associated with other forms of therapy). Further, when the detectable agent comprises a diagnostic radionuclide, the conjugates may be used to select patients that may benefit most from the therapy or to monitor response to therapy—a step toward more personalized medicine for cancer patients.

In certain other embodiments, the detectable agent comprises a fluorescent group, and the resulting conjugates may be used, e.g., for assays designed to identify and quantify the concentration of GRP78 on cell surfaces.

In certain embodiments, the detectable agent is not DOTA or fluorescein.

### Chelating Groups and Radionuclides

In certain embodiments of the invention, the detectable agent comprises a chelating group. As used herein, a “chelating group” is a group that can include a detectable group, e.g., a radionuclide (e.g., a metallic radioisotope). Any suitable chelating group can be employed. Suitable chelating groups are disclosed, e.g., in Rockey et al., Bioorganic & Medicinal Chemistry 19 (2011) 4080-4090; Poster Sessions, Proceedings of the 46th Annual Meeting, J. Nuc. Med., p. 316, No. 1386; Scientific Papers, Proceedings of the 46th Annual Meeting, J. Nuc. Med., p. 123, No. 499; Scientific Papers, Proceedings of the 46th Annual Meeting, J. Nuc. Med., p. 102, No. 413; Scientific Papers, Proceedings of the 46th Annual Meeting, J. Nuc. Med., p. 102, No. 414; Scientific Papers, Proceedings of the 46th Annual Meeting, J. Nuc. Med., p. 103, No. 415; Poster Sessions, Proceedings of the 46th Annual Meeting, J. Nuc. Med., p. 318, No. 1396; Poster Sessions, Proceedings of the 46th Annual Meeting, J. Nuc. Med., p. 319, No. 1398; M. Moi et al., J. Amer. Chem., Soc., 49, 2639 (1989); S. V. Deshpande et al., J. Nucl. Med., 31, 473 (1990); G. Kuser et al., Bioconj. Chem., 1, 345 (1990); C. J. Broan et al., J. C. S. Chem. Comm., 23, 1739 (1990); C. J. Anderson et al., J. Nucl. Med. 36, 850 (1995); U.S. Pat. No. 5,739,313; and U.S. Pat. No. 6,004,533. Additionally, certain chelating groups are available from Macrocyclics (https://macrocyclics.com/shop/) and are listed in Table 1 below.

In certain embodiments, the detectable agent comprises a chelating group selected from:

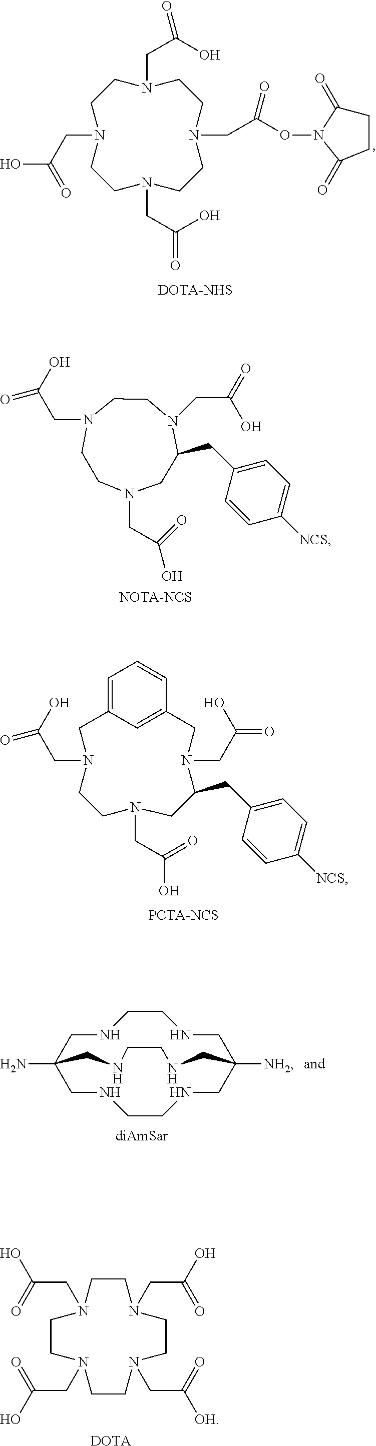

In certain embodiments, the detectable agent comprises DOTA.

In certain embodiments, the detectable agent does not comprise DOTA.

Conjugates of the invention, e.g., radiolabeled conjugates of formula I, are useful as imaging agents for imaging cells and tissues that include GRP78, as well as for therapy. Accordingly, in certain embodiments, the invention also provides conjugates of formula I, wherein D comprises a chelating group that includes one or more detectable radionuclides (e.g., one or more metallic radionuclides, e.g., emits a signal). Methods for making such detectable agents are known to the art worker. Such conjugates can be useful to image tissues expressing GRP78 in vivo or in vitro or for therapeutic purposes.

As used herein, a “detectable radionuclide” is any suitable radionuclide (i.e., a radioisotope) useful in an imaging procedure, e.g., a diagnostic procedure, in vivo or in vitro, or for, e.g., therapy, e.g., cancer therapy. Suitable detectable radionuclides include metallic radionuclides (i.e., metallic radioisotopes).

Suitable metallic radionuclides (i.e., metallic radioisotopes or metallic paramagnetic ions) include Antimony-124, Antimony-125, Arsenic-74, Barium-103, Barium-140, Beryllium-7, Bismuth-206, Bismuth-207, Cadmium-109, Cadmium-115m, Calcium-45, Cerium-139, Cerium-141, Cerium-144, Cesium-137, Chromium-51, Cobalt-55, Cobalt-56, Cobalt-57, Cobalt-58, Cobalt-60, Cobalt-64, Copper-64, Copper-67, Erbium-169, Europium-152, Gallium-64, Gallium-68, Gadolinium-153, Gadolinium-157 Gold-195, Gold-199, Hafnium-175, Hafnium-175-181, Holmium-166, Indium-110, Indium-111, Iridium-192, Iron-55, Iron-59, Krypton-85, Lead-210, Manganese-54, Mercury-197, Mercury-203, Molybdenum-99, Neodymium-147, Neptunium-237, Nickel-63, Niobium-95, Osmium-185+191, Palladium-103, Platinum-195m, Praseodymium-143, Promethium-147, Protactinium-233, Radium-226, Rhenium-186, Rhenium-188, Rubidium-86, Ruthenium-103, Ruthenium-106, Scandium-44, Scandium-46, Selenium-75, Silver-110m, Silver-111, Sodium-22, Strontium-85, Strontium-89, Strontium-90, Sulfur-35, Tantalum-182, Technetium-99m, Tellurium-125, Tellurium-132, Thallium-204, Thorium-228, Thorium-232, Thallium-170, Tin-113, Tin-114, Tin-117m, Titanium-44, Tungsten-185, Vanadium-48, Vanadium-49, Ytterbium-169, Yttrium-86, Yttrium-88, Yttrium-90, Yttrium-91, Zinc-65, and Zirconium-95.

In certain embodiments, the radionuclide is Gallium-68, Copper-64 or Yttrium-90.

In certain embodiments, the radionuclide is Gallium-68.

In certain embodiments, the radionuclide is not Gallium-68.

In certain embodiments, D is not a DOTA chelating group including Gallium-68.

In some embodiments of the invention, the chelating group can include more than one independently selected metallic radioisotope. In some embodiments, the detectable chelating group can include 2 to about 10, 2 to about 8, 2 to about 6, or 2 to about 4 independently selected metallic radioisotopes.

### Fluorescent Groups

In certain embodiments, the detectable agent comprises a fluorescent group, which may also be called a “fluorescent tag” or a “fluorophore”. Thus, the resulting conjugates may be used, e.g., for assays designed to identify and quantify the concentration of GRP78 on cell surfaces.

A fluorophore is a molecule that absorbs light (i.e. excites) at a characteristic wavelength and emits light (i.e. fluoresces and emits a signal) at a second lower-energy wavelength. The detectable agent may include, but is not limited to, one or more of the following fluorescent groups: fluorescein, tetrachlorofluorescein, hexachlorofluorescein, tetramethylrhodamine, rhodamine, cyanine-derivative dyes, Texas Red, Bodipy, and Alexa dyes. Characteristic absorption and emission wavelengths for each of these are well known to those of skill in the art.

In certain embodiments, the fluorescent group is fluorescein.

In certain embodiments, the fluorescent group is not fluorescein.

In certain embodiments, the fluorophore is one or more of the fluorophores listed in Table 2.

TABLE 2

| | Excitation | Emission |
|---|---|---|
| Probe | (nm) | (nm) |
| Hydroxycoumarin | 325 | 386 |
| Alexa fluor | 325 | 442 |
| Aminocoumarin | 350 | 445 |
| Methoxycoumarin | 360 | 410 |
| Cascade Blue | (375); 401 | 423 |
| Pacific Blue | 403 | 455 |
| Pacific Orange | 403 | 551 |
| Lucifer yellow | 425 | 528 |
| Alexa fluor 430 | 430 | 545 |
| NBD | 466 | 539 |
| R-Phycoerythrin (PE) | 480; 565 | 578 |
| PE-Cy5 conjugates | 480; 565; 650 | 670 |
| PE-Cy7 conjugates | 480; 565; 743 | 767 |
| Red 613 | 480; 565 | 613 |
| PerCP | 490 | 675 |
| Cy2 | 490 | 510 |
| TruRed | 490, 675 | 695 |
| FluorX | 494 | 520 |
| Fluorescein | 495 | 519 |
| FAM | 495 | 515 |
| BODIPY-FL | 503 | 512 |
| TET | 526 | 540 |
| Alexa fluor 532 | 530 | 555 |
| HEX | 535 | 555 |
| TRITC | 547 | 572 |
| Cy3 | 550 | 570 |
| TMR | 555 | 575 |
| Alexa fluor 546 | 556 | 573 |
| Alexa fluor 555 | 556 | 573 |
| Tamara | 565 | 580 |
| X-Rhodamine | 570 | 576 |
| Lissamine Rhodamine B | 570 | 590 |
| ROX | 575 | 605 |
| Alexa fluor 568 | 578 | 603 |
| Cy3.5 581 | 581 | 596 |
| Texas Red | 589 | 615 |
| Alexa fluor 594 | 590 | 617 |
| Alexa fluor 633 | 621 | 639 |
| LC red 640 | 625 | 640 |
| Allophycocyanin (APC) | 650 | 660 |
| Alexa fluor 633 | 650 | 688 |
| APC-Cy7 conjugates | 650; 755 | 767 |
| Cy5 | 650 | 670 |
| Alexa fluor 660 | 663 | 690 |
| Cy5.5 | 675 | 694 |
| LC red 705 | 680 | 710 |
| Alexa fluor 680 | 679 | 702 |
| Cy7 | 743 | 770 |
| IRDye 800 CW | 774 | 789 |

In certain in vivo embodiments, the fluorophore emits in the near infrared range, such as in the 650-900 nm range. (Weissleder et al., “Shedding light onto live molecular targets, Nature Medicine, 9:123-128 (2003)).

In certain embodiments, D is a pharmaceutically active agent. The pharmaceutically active agent may have activity when it is linked to the peptide or may become active when the linking group is hydrolyzed and the pharmaceutically active agent is released from the remainder of the conjugate. In certain embodiments, the pharmaceutically active agent is a chemotherapeutic agent.

### Certain Conjugate Embodiments

In certain embodiments, a conjugate of formula (I) is selected from:

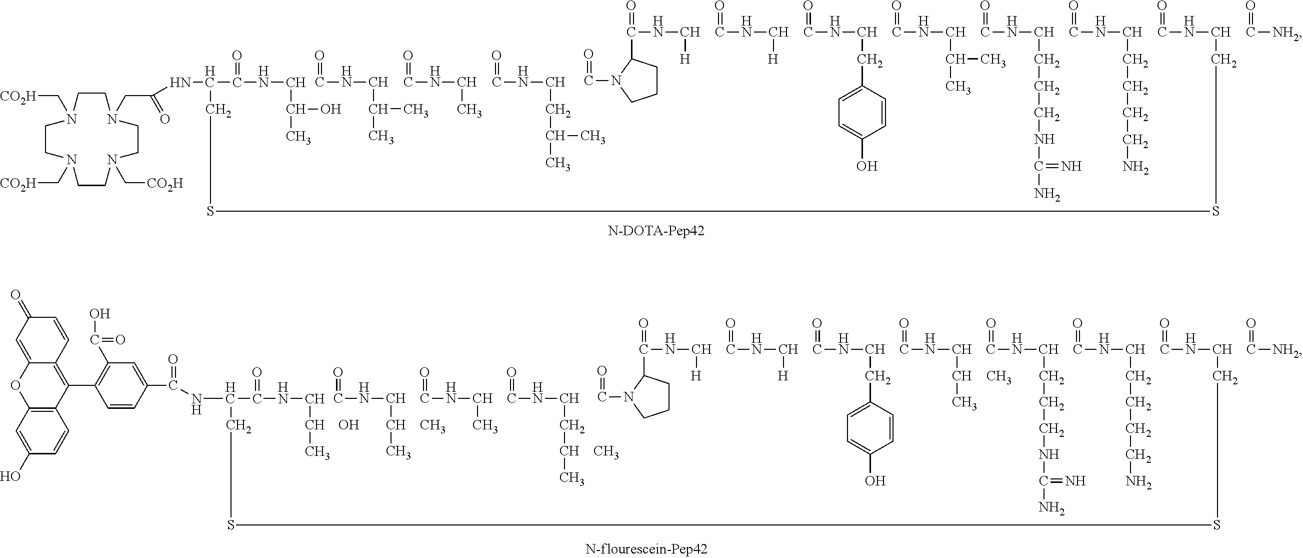

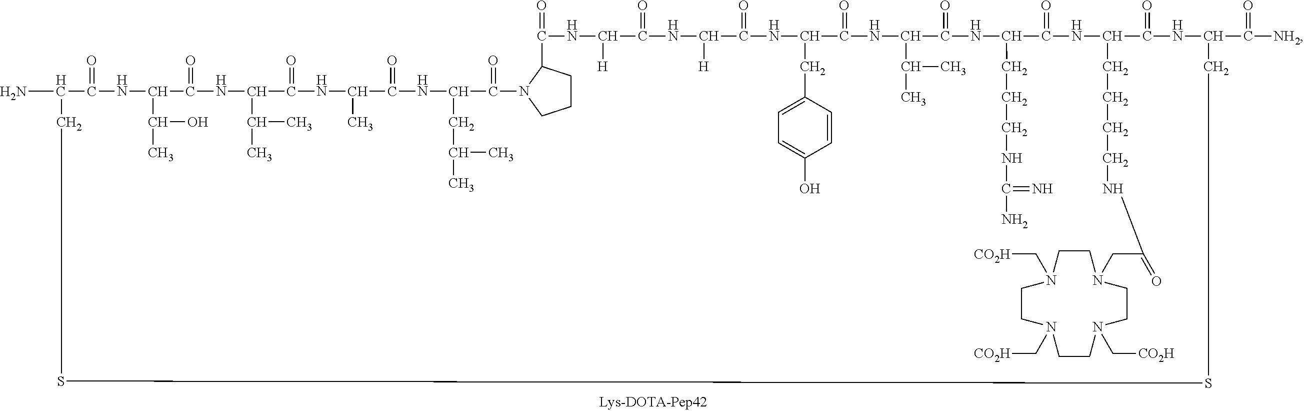 [SMILES: CC(C)CC1NC(=O)C(C)NC(=O)C(C(C)C)NC(=O)C(C(C)O)NC(=O)C(N)CSSCC(C(N)=O)NC(=O)C(CCCCNC(=O)CN2CCN(CC(=O)O)CCN(CC(=O)O)CCN(CC(=O)O)CC2)NC(=O)C(CCCNC(=N)N)NC(=O)C(C(C)C)NC(=O)C(Cc2ccc(O)cc2)NC(=O)CNC(=O)CNC(=O)C2CCCN2C1=O]

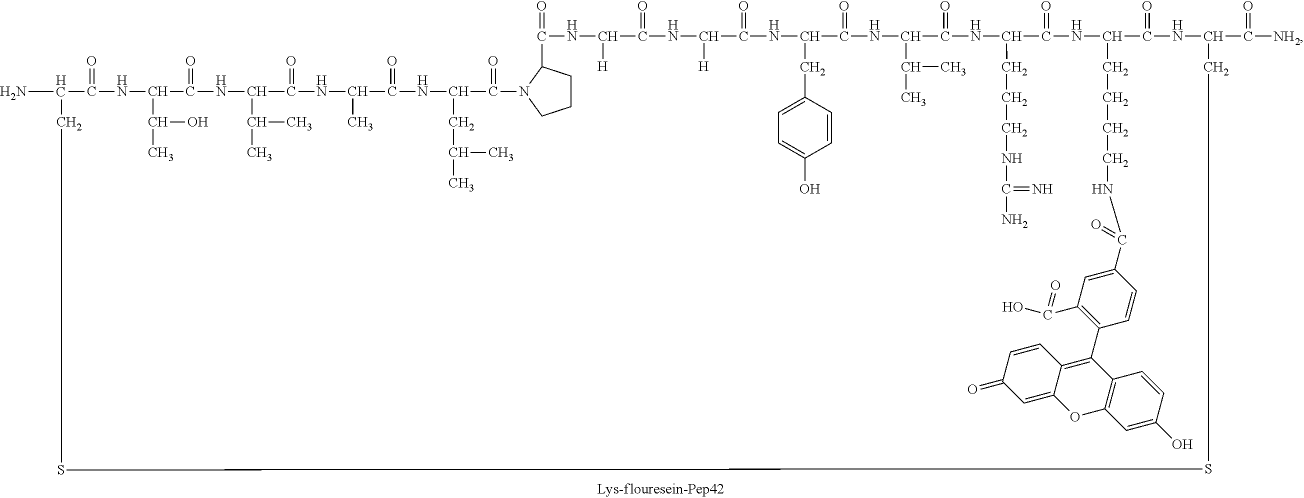 [SMILES: CC(C)CC1NC(=O)C(C)NC(=O)C(C(C)C)NC(=O)C(C(C)O)NC(=O)C(N)CSSCC(C(N)=O)NC(=O)C(CCCCNC(=O)c2ccc(-c3c4ccc(=O)cc-4oc4cc(O)ccc34)c(C(=O)O)c2)NC(=O)C(CCCNC(=N)N)NC(=O)C(C(C)C)NC(=O)C(Cc2ccc(O)cc2)NC(=O)CNC(=O)CNC(=O)C2CCCN2C1=O]

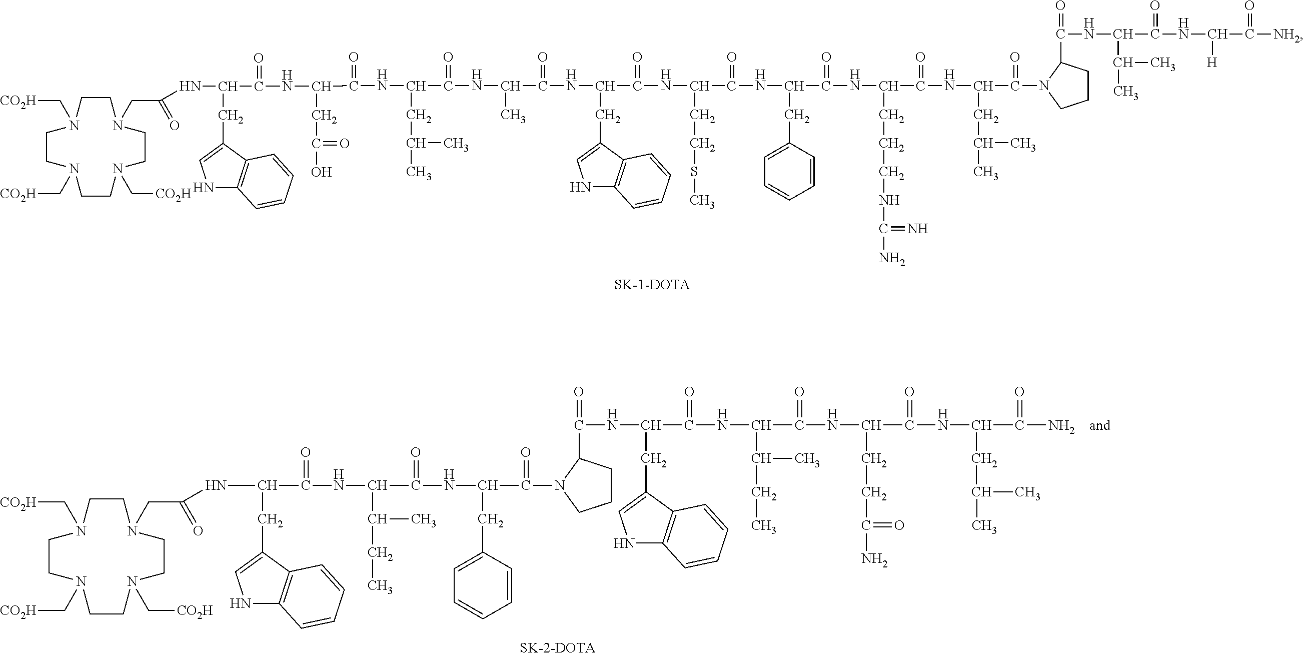 [SMILES: CSCCC(NC(=O)C(Cc1c[nH]c2ccccc12)NC(=O)C(C)NC(=O)C(CC(C)C)NC(=O)C(CC(=O)O)NC(=O)C(Cc1c[nH]c2ccccc12)NC(=O)CN1CCN(CC(=O)O)CCN(CC(=O)O)CCN(CC(=O)O)CC1)C(=O)NC(Cc1ccccc1)C(=O)NC(CCCNC(=N)N)C(=O)NC(CC(C)C)C(=O)N1CCCC1C(=O)NC(C(=O)NCC(N)=O)C(C)C ; CCC(C)C(NC(=O)C(Cc1c[nH]c2ccccc12)NC(=O)C1CCCN1C(=O)C(Cc1ccccc1)NC(=O)C(NC(=O)C(Cc1c[nH]c2ccccc12)NC(=O)CN1CCN(CC(=O)O)CCN(CC(=O)O)CCN(CC(=O)O)CC1)C(C)CC)C(=O)NC(CCC(N)=O)C(=O)NC(CC(C)C)C(N)=O ; "and"]

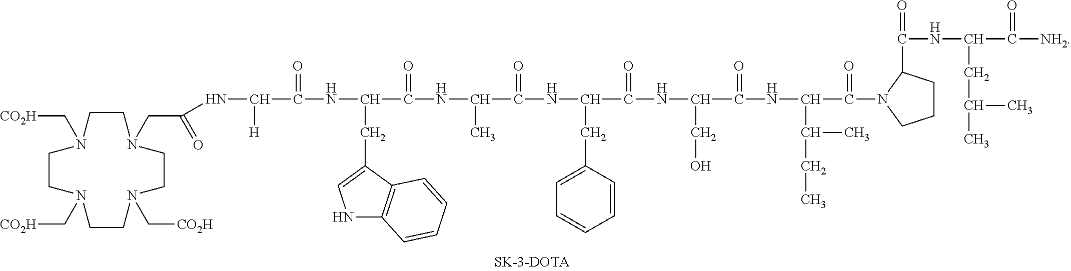 [SMILES: CCC(C)C(NC(=O)C(CO)NC(=O)C(Cc1ccccc1)NC(=O)C(C)NC(=O)C(Cc1c[nH]c2ccccc12)NC(=O)CNC(=O)CN1CCN(CC(=O)O)CCN(CC(=O)O)CCN(CC(=O)O)CC1)C(=O)N1CCCC1C(=O)NC(CC(C)C)C(N)=O]

Conjugates of formula (I) may be synthesized using methods known in the art or using methods described herein (e.g., Example 1). For example, the peptides described herein may be generated using solid phase synthesis and subsequently characterized using, e.g., HPLC, LCMS or Mass Spec. A detectable agent (D), such as chelators or fluorophores, may be conjugated to the peptides as described herein via a linking group (X), and subsequently characterized using, e.g., HPLC, LCMS or Mass Spec. The conjugate may then be purified using, e.g., HPLC.

Certain embodiments of the invention provide a GRP78 targeting peptide comprising amino acid sequence GWAFSIPL (SEQ ID NO:4).

Certain embodiments of the invention provide a GRP78 targeting peptide consisting of amino acid sequence GWAFSIPL (SEQ ID NO:4).

As used herein, the following definitions are used, unless otherwise described: halo is fluoro, chloro, bromo, or iodo. Alkyl, alkoxy, alkenyl, alkynyl, etc. denote both straight and branched groups; but reference to an individual radical such as propyl embraces only the straight chain radical, a branched chain isomer such as isopropyl being specifically referred to. Aryl denotes a phenyl radical or an ortho-fused bicyclic carbocyclic radical having about nine to ten ring atoms in which at least one ring is aromatic. Heteroaryl encompasses a radical of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms comprising one to four heteroatoms each selected from the group consisting of non-peroxide oxygen, sulfur, and N(X).

Specific values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

### Treatment and Diagnostic Methods

Certain embodiments of the invention provide a pharmaceutical composition comprising a conjugate of formula I and a pharmaceutically acceptable excipient.

Certain embodiments of the invention provide a method for treating or preventing cancer in an animal (e.g., a human) comprising administering a therapeutically effective amount of a conjugate (e.g., comprising a therapeutic radionuclide) of formula I to the animal.

The terms “treat” and “treatment” refer to both therapeutic treatment and prophylactic or preventative measures, wherein the object is to prevent or slow down (lessen) an undesired physiological change or disorder, such as the growth, development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, head and neck cancer, and melanoma.

The invention also provides a conjugate of formula I for use in medical therapy.

The invention also provides a conjugate of formula I for the prophylactic or therapeutic treatment of cancer.

The invention also provides the use of a conjugate of formula (I) to prepare a medicament for treating cancer in an animal (e.g. a mammal such as a human).

In certain embodiments, the cancer is melanoma, breast cancer or prostate cancer.

In certain embodiments, the cancer is melanoma.

Certain embodiments of the invention provide a method of detecting a GRP78 molecule, comprising contacting a cell with a conjugate of formula (I). In certain embodiments, the detectable agent comprises a chelating group labeled with a radionuclide. In certain embodiments, the detectable agent comprises a fluorescent group. In certain embodiments, the method further comprises quantifying the concentration of GRP78 on the surface of the cell by measuring a signal from the detectable agent (e.g., a fluorescent signal or a radioactive signal). Methods of measuring a signal from a detectable agent, such as a radioactive signal or fluorescent signal, are known in the art; for example, such methods may include flow cytometery or confocal microscopy for detecting a fluorescent signal or the use of a scintillation counter to measure a radioactive signal. Accordingly, in certain embodiments, the method further comprises quantifying the concentration of GRP78 on the surface of the cell by measuring a signal from the detectable agent using flow cytometry or confocal microscopy. In certain embodiments, the method further comprises quantifying the concentration of GRP78 on the surface of the cell by measuring a signal from the detectable agent using a scintillation counter.

As described herein, the extent of GRP78 expression on a cell surface is also associated with the progression and stage of cancer (e.g., melanoma). Accordingly, in certain embodiments, the invention relates to methods of using the conjugates for in vitro, in situ, and in vivo diagnosis of cancer (such as melanoma), as well as for determining the effectiveness of a cancer treatment.

Certain embodiments of the invention provide a method of detecting cancer cells in a test tissue sample, comprising contacting the test sample with a conjugate of formula (I) and measuring a signal from the detectable agent (e.g., a radioactive signal or fluorescent signal), wherein a signal greater than a signal from a non-cancerous control sample indicates the presence of cancer cells in the test tissue sample. In certain embodiments, the signal from the test sample is 1-100% greater than the signal from the control sample. In certain embodiments, the signal from the test sample is 10%, 20%, 30%, 40%, 50%, 60%, 70%, 80%, or 90% greater than the signal from the control sample. In certain embodiments, the signal from the detectable agent is measured using a scintillation counter, confocal microscopy or flow cytometry.

Certain embodiments of the invention provide an in vivo method of detecting cancer in an animal (e.g., a human patient), comprising administering a conjugate of formula (I) to the animal and measuring a signal (e.g., a radioactive signal or fluorescent signal emitting in the near infrared range) from the detectable agent, wherein a signal greater than a signal from a control animal without cancer indicates the animal has cancer. In certain embodiments, the signal from the animal is 1-100% greater than the signal from the control animal. In certain embodiments, the signal from the animal is 10%, 20%, 30%, 40%, 50%, 60%, 70%, 80%, or 90% greater than the signal from the control animal. In certain embodiments of the invention, the signal from the detectable agent is measured using PET imaging or MRI.

Certain embodiments of the invention provide a method for determining the effectiveness of a cancer therapy in an animal (e.g., a human patient), comprising
1) administering a conjugate of formula (I) to the animal;
2) obtaining a first test sample from the animal and measuring a first signal (e.g., a radioactive signal or fluorescent signal) from the detectable agent;
3) administering a cancer therapy;
4) administering a conjugate of formula (I) to the animal;
5) obtaining a second test sample from the animal and measuring a second signal (e.g., a radioactive signal or fluorescent signal) from the detectable agent; and
6) comparing the first signal with the second signal, wherein the cancer therapy is effective if the second signal is less than the first signal.

In certain embodiments, the first and second signals are measured using a scintillation counter, confocal microscopy or flow cytometry.

Certain embodiments of the invention provide a method for determining the effectiveness of a cancer therapy in an animal (e.g., a human patient), comprising
1) administering a conjugate of formula (I) to the animal and measuring a first signal (e.g., a radioactive signal or fluorescent signal emitting in the near infrared range) from the detectable agent;
2) administering a cancer therapy;
3) administering a conjugate of formula (I) to the animal and measuring a second signal (e.g., a radioactive signal or fluorescent signal emitting in the near infrared range) from the detectable agent; and
4) comparing the first signal with the second signal, wherein the cancer therapy is effective if the second signal is less than the first signal.

In certain embodiments, the second signal is 1-100% less than the first signal. In certain embodiments, the first signal is 10%, 20%, 30%, 40%, 50%, 60%, 70%, 80%, or 90% less than the first signal. In certain embodiments of the invention, the signal from the detectable agent is measured using PET imaging or by MRI. In certain embodiments of the invention, the signal from the detectable agent is measured using PET imaging.

Certain embodiments of the invention provide a method of treating a cancer in an animal (e.g., a human patient), comprising
1) administering a conjugate of formula (I) to the animal and measuring a first signal (e.g., a radioactive signal or fluorescent signal emitting in the near infrared range) from the detectable agent;
2) administering a cancer therapy;
3) administering a conjugate of formula (I) to the animal and measuring a second signal (e.g., a radioactive signal or fluorescent signal emitting in the near infrared range) from the detectable agent; and
4) comparing the first signal with the second signal, wherein the cancer therapy is effective if the second signal is less than the first signal.

Certain embodiments of the invention provide a kit comprising:
1) a conjugate of formula (I); and
2) instructions for administering the conjugate to an animal.

Certain embodiments of the invention provide a kit comprising:
1) a conjugate of formula (I);
2) instructions for loading a radionuclide into the conjugate to generate a radiolabeled conjugate; and
3) instructions for administering the radiolabeled conjugate to an animal.

Certain embodiments of the invention provide a kit comprising:
1) a conjugate of formula (I);
2) a radionuclide;
3) instructions for loading the radionuclide into the conjugate to generate a radiolabeled conjugate; and
4) instructions for administering the radiolabeled conjugate to an animal.

### Administration

The conjugates of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present conjugates may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the conjugates may be combined with one or more excipients and used in the form of ingestible tablets, buccal tablets, troches, capsules, elixirs, suspensions, syrups, wafers, and the like. Such compositions and preparations should contain at least 0.1% of conjugates. The percentage of the compositions and preparations may, of course, be varied and may conveniently be between about 2 to about 60% of the weight of a given unit dosage form. The amount of the conjugates in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the conjugates, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the conjugates may be incorporated into sustained-release preparations and devices.

The conjugates may also be administered intravenously or intraperitoneally by infusion or injection. Solutions of the conjugates can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

In certain embodiments, a conjugate of formula (I), wherein the detectable group comprises a chelating group labeled with a radionuclide, is formulated for administration by infusion.

The pharmaceutical dosage forms suitable for injection or infusion can include sterile aqueous solutions or dispersions or sterile powders comprising the conjugates which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the conjugates in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the conjugates plus any additional desired ingredient present in the previously sterile-filtered solutions.

For topical administration, the present conjugates may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present conjugates can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the conjugates of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the conjugates of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the conjugates, or derivative thereof, required for use in treatment will vary with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Conjugates of the invention can also be administered in combination with other therapeutic agents, for example, other agents that are useful for the treating cancer. Examples of such agents include chemotherapeutic agents. Accordingly, one embodiment the invention also provides a composition comprising a conjugate of formula I, at least one other therapeutic agent, and a pharmaceutically acceptable diluent or carrier. The invention also provides a kit comprising a conjugate of formula I, at least one other therapeutic agent, packaging material, and instructions for administering the conjugate of formula I and the other therapeutic agent or agents to an animal to treat cancer.

The invention will now be illustrated by the following non-limiting Example.

### Example 1

### Abstract

GRP78 is a 78 kDa molecular chaperone that resides primarily in the endoplasmic reticulum however emerging evidences have shown translocation of GRP78 on the cell surface in the tumor microenvironment (Amy. S. Lee Cancer Res (2007); 77:3496-3499). As described herein the potential of GRP78 targeted peptides for molecular imaging and targeted radionuclide therapy were evaluated. Two sets of GRP78 targeted peptides were synthesized. The PEP42 analogues (N-DOTA/flourescein-PEP42 and Lys-DOTA/flourescein-PEP42) are 13-mer cyclized peptides in which the position of DOTA was varied. SK peptides, which have hydrophobic amino acids at even positions and the DOTA conjugated at the N-terminus, were also evaluated. The DOTA conjugates of these peptides were successfully labeled with ⁶⁸Ga in 0.1 M acetate buffer at 99° C. with 12 min incubation. Difference in the binding potential of these peptides are evaluated using B16-tumor tissue cross sections and 1-LN prostate cancer cells.

### Introduction

GRP78 is member of the heat shock protein family and a molecular chaperone that has long been considered to be confined to the endoplasmic reticulum, where it plays a major role in mediating appropriate protein folding and the unfolded protein response (UPR) in the cell (J. Li et. al. Cell Death and Differentiation (2008) 15, 1460-1471). Emerging evidences have demonstrated that GRP78 is exported from the internal organelle to the surface of the cell under conditions of stress (e.g., oxidative stress) (Amy. S. Lee Cancer Res (2007); 77:3496-3499). Although the mechanism and reasons for the protein to be present on the surface of cells is not well understood, it is increasingly being related to intracellular and extra cellular stresses that are associated with tumor microenvironment. The extent of GRP78 expression on cell surface is also associated with progression and stage of cancer (e.g., melanoma) (Liquing Zhuang et al. Histopathology (2009), 54, 462-470). Cell surface re-localization also plays a role in drug resistance.

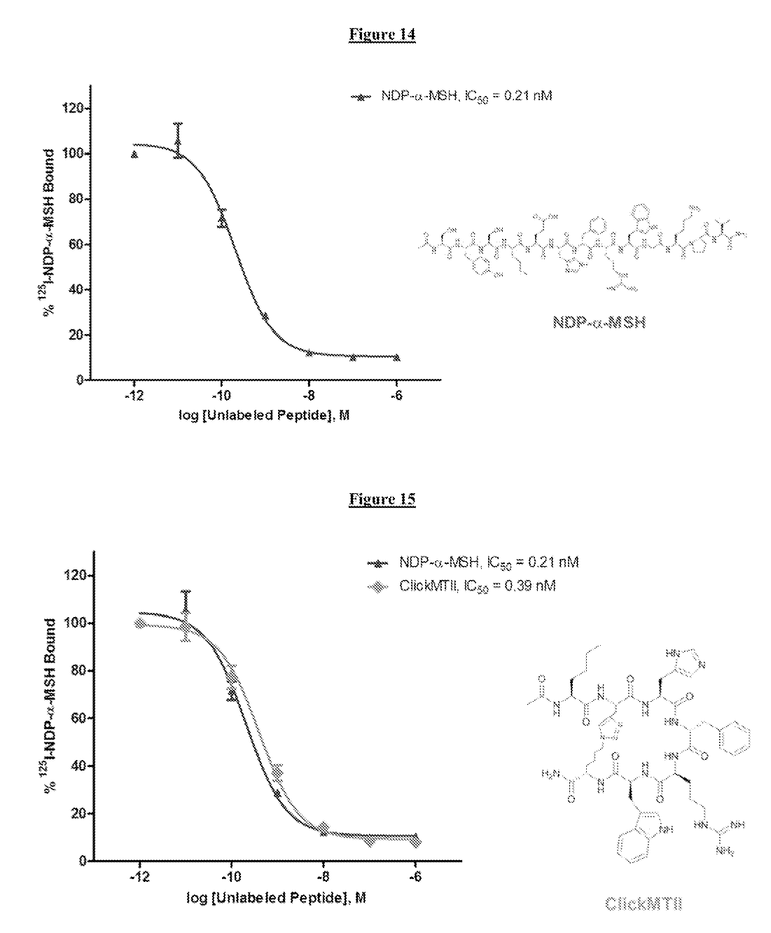

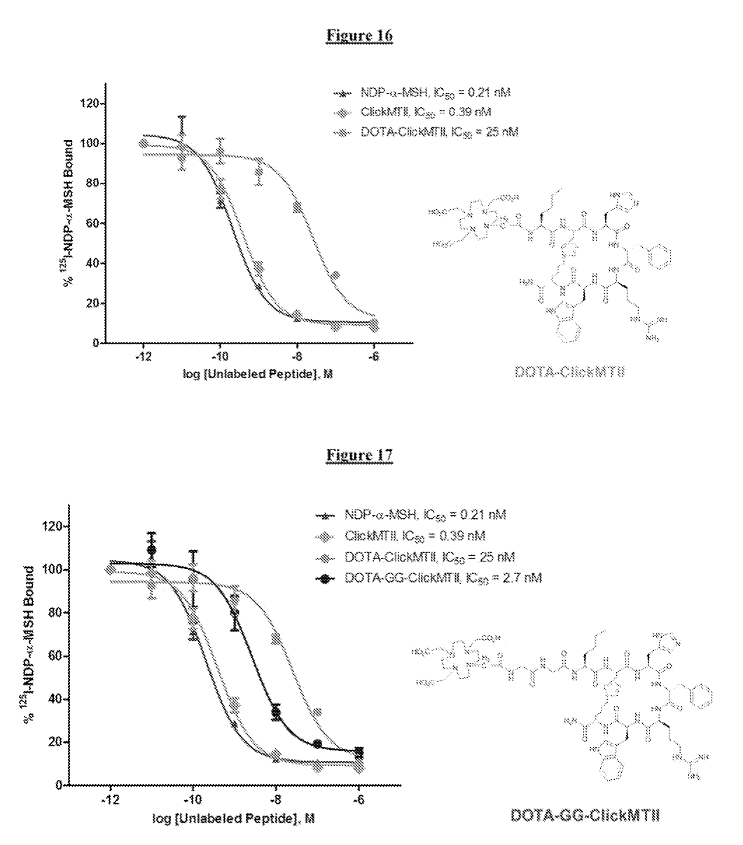

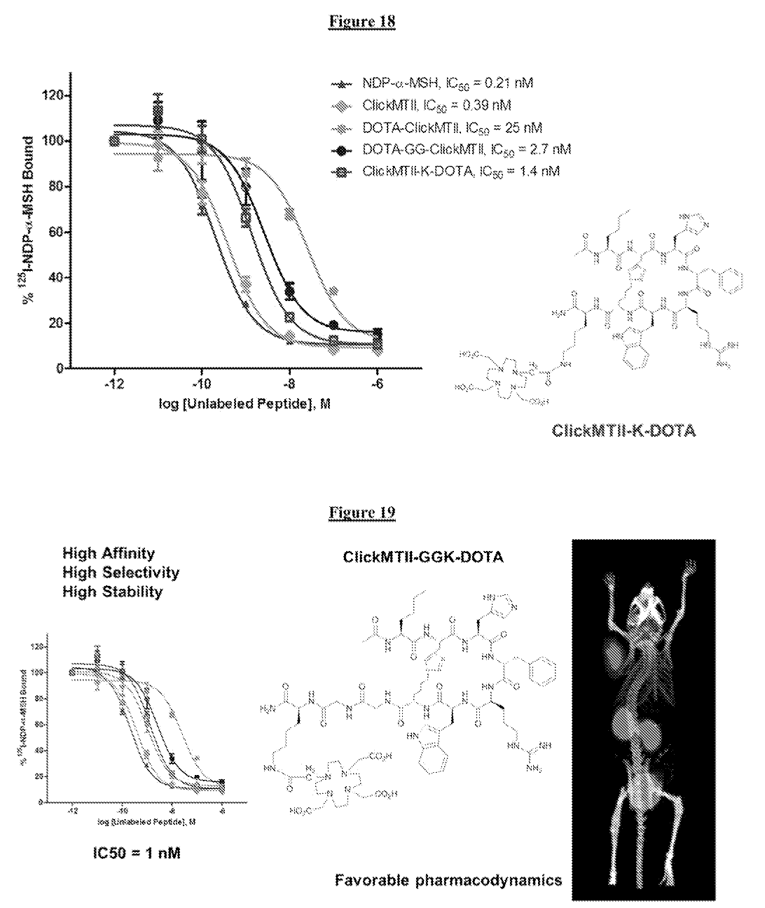

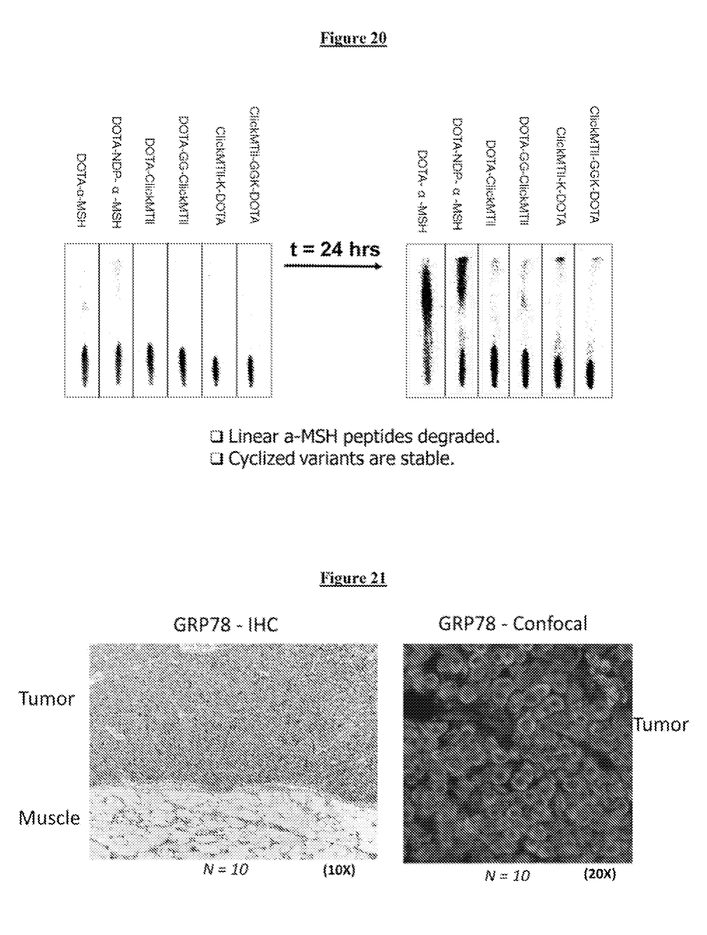

As described herein, GRP78 is a potential target for molecular imaging (e.g., PET imaging) and targeted radionuclide therapy (e.g., cancer therapy, such as melanoma) using molecules, such as peptides, that are designed to bind with high affinity to it. Thus, molecules (small amino acid sequences like PEP42 (Ying Liu et. al. Molecular Pharmaceutics (2007)) and SK-2) and their DOTA/fluorescein conjugates have been synthesized to study their affinity and specificity towards GRP78. Based on the initial biological evaluation of these GRP78 targeted peptides, further modification of the structure of these peptides may be performed to increase their stability, binding affinity and specificity towards GRP78 (e.g., by modifying peptide sequence, type of chelating/fluorescent group, linking group, or attachment point of the chelating/fluorescent group as described herein; see examples shown in FIGS. 14-19).

### Materials and Methods

GRP78 Targeted Peptides and their Conjugates

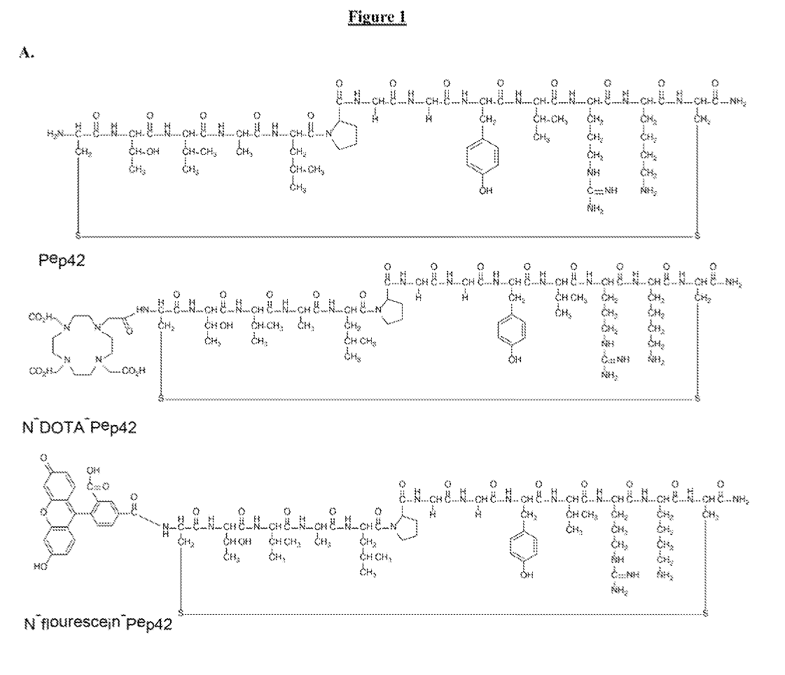

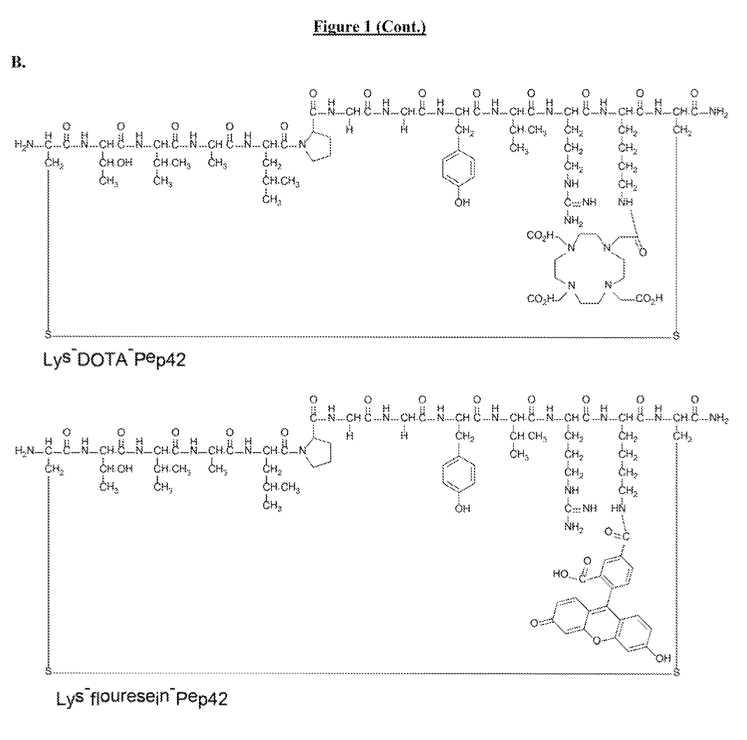

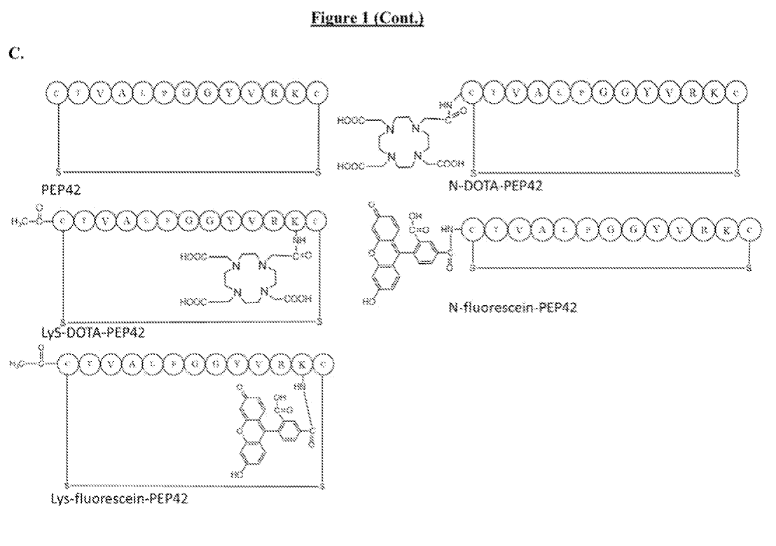

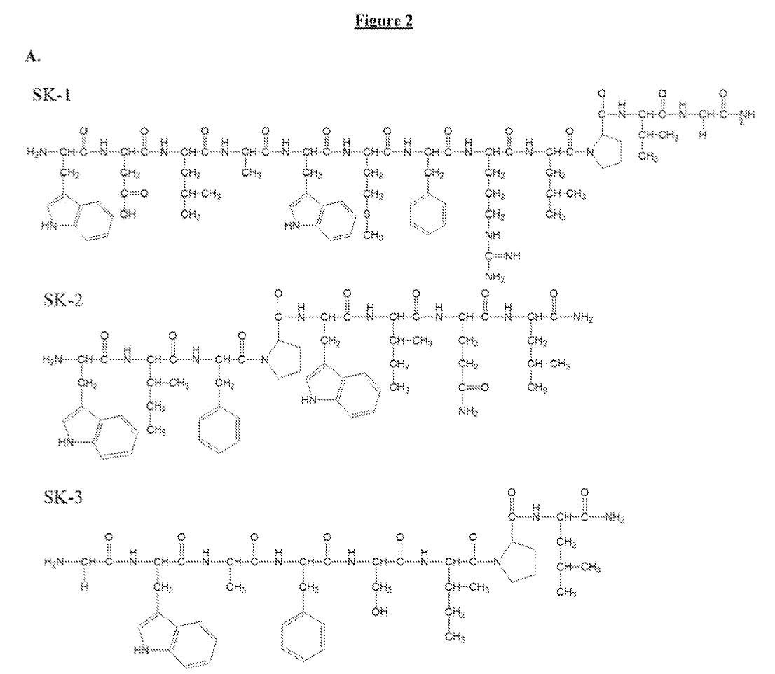

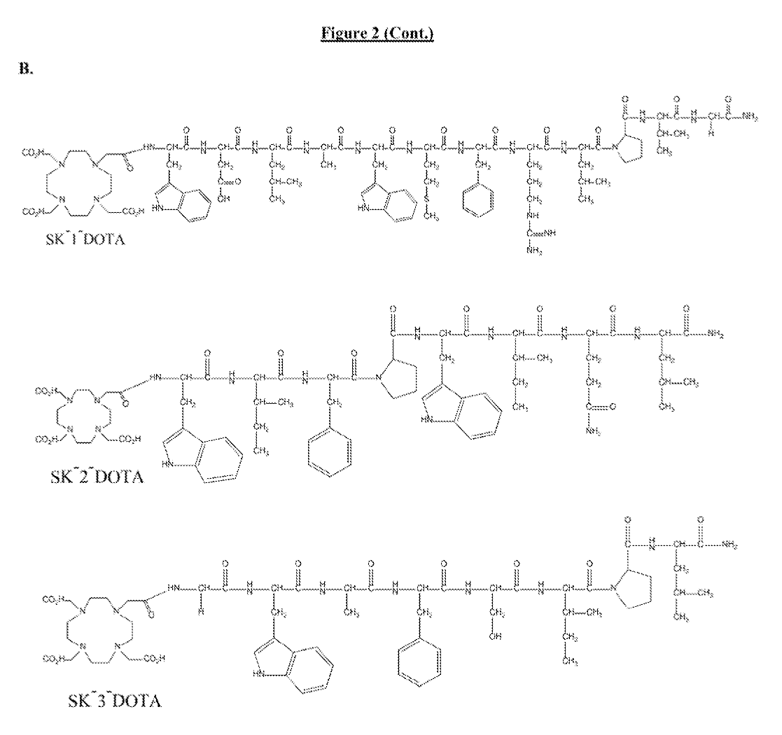

Seven GRP78 targeted peptide derivatives with micromolar affinity (8 to 13-mer in length) were synthesized based on previous studies in which peptides with were selected by bacteriophage techniques for other applications (J. Li et. al. Cell Death and Differentiation (2008) 15, 1460-1471; Liquing Zhuang et. Al Histopathology (2009), 54, 462-470; and Ying Liu et. al. Molecular Pharmaceutics (2007)). Three linear and four cyclized derivatives were modified with a DOTA chelator and a fluorescent (fluorescein) moiety (see, FIGS. 1-2). DOTA conjugates were labeled with Ga-68 in acetate buffer at 99° C. Specific methods are included below.

PEP42.

PEP42 is a 13-mer cyclic peptide that is shown to bind with GRP78 in cell free binding assays (Ying Liu et. al. Molecular Pharmaceutics (2007) (FIG. 1A). The PEP42 peptide has an amino acid sequence: CTVALPGGYVRKC (SEQ ID NO:1).

N-DOTA-PEP42.

DOTA was conjugated to the N-terminus of PEP42 via an amide bond (FIGS. 1A and 1C). DOTA conjugated peptides can be labeled with ⁶⁸Ga. ⁶⁸Ga was obtained using an IGG100 generator (Eckert Ziegler, GmBH, Berlin, De) and peptides labeled by published procedures. Briefly, ⁶⁸Ga (˜900 MBq) was eluted with 10 mL of 0.1 M HCl (2 mL per minute) and adsorbed to a cation exchange (Telos SPE Columns). The column is air dried and ⁶⁸Ga is eluted with a mixture of 500 μL of 5.5 M HCl and 12.5 μL of 5M NaCl directly in a glass vial containing known amount of DOTA-peptide dissolved in 4 mL acetic acid-acetate buffer (pH=3.8). This solution was then heated at 100° C. for 12 min. The radiolabeling efficiency was obtained by radio HPLC (see, FIG. 3A).

N-FAM PEP42.

FAM is fluorophore that can be conjugated to the N-terminus or any free amine via an amide bond (FIGS. 1A and 1C). This PEP42 derivative was synthesized to evaluate the binding affinity of PEP42 with GRP78 by microscopy. B16 tumor tissue cross section was incubated with 20 μM of N-FAM PEP42 for an hour and the unbound peptide was washed away by subsequent PBS washes. The confocal image shows that the fluorophore binding to cells in the tissue sample (FIG. 3B). However fluorescein cyclized derivatives were internalized and binding affinity did not depend on the position of DOTA/fluorescein.

SK-2.

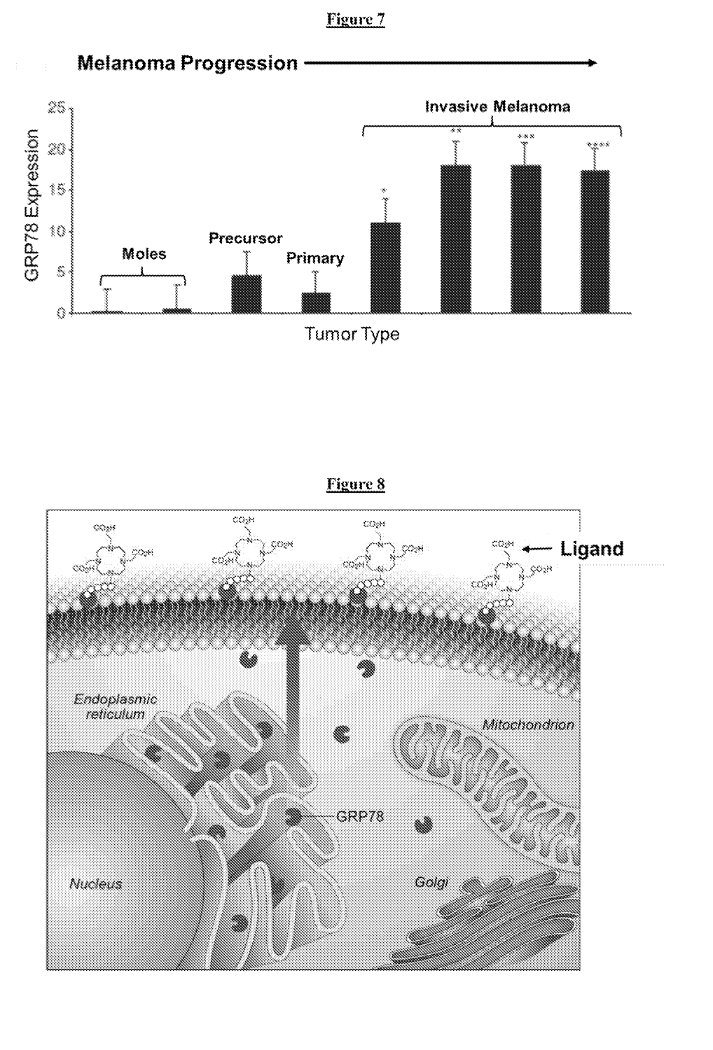

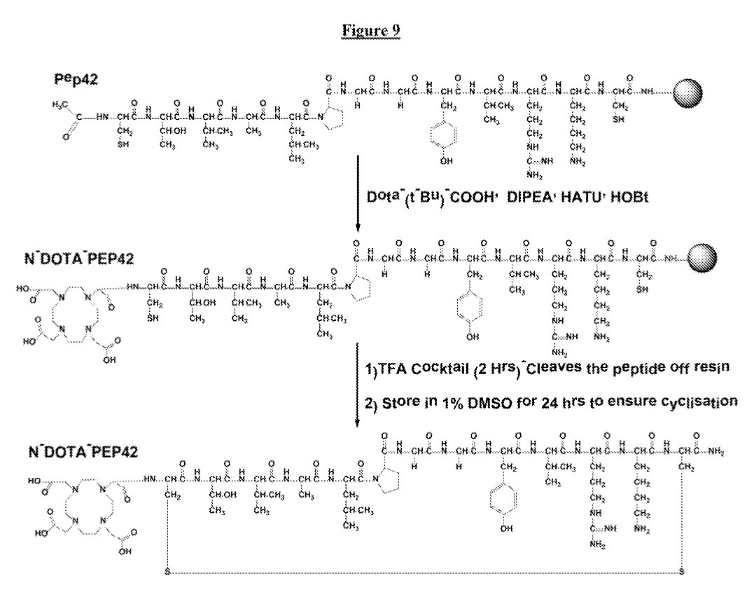

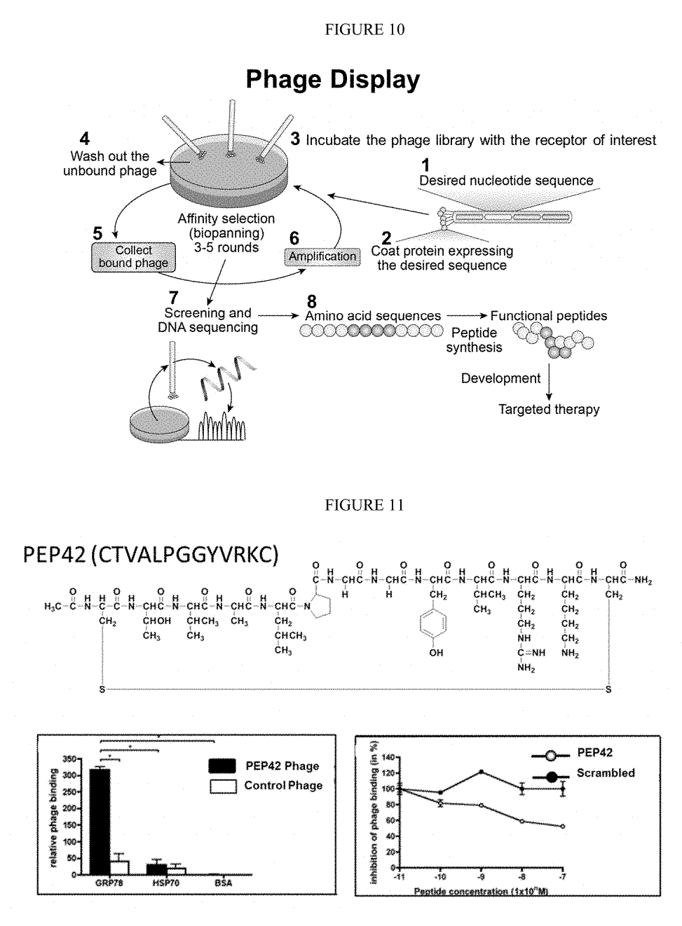

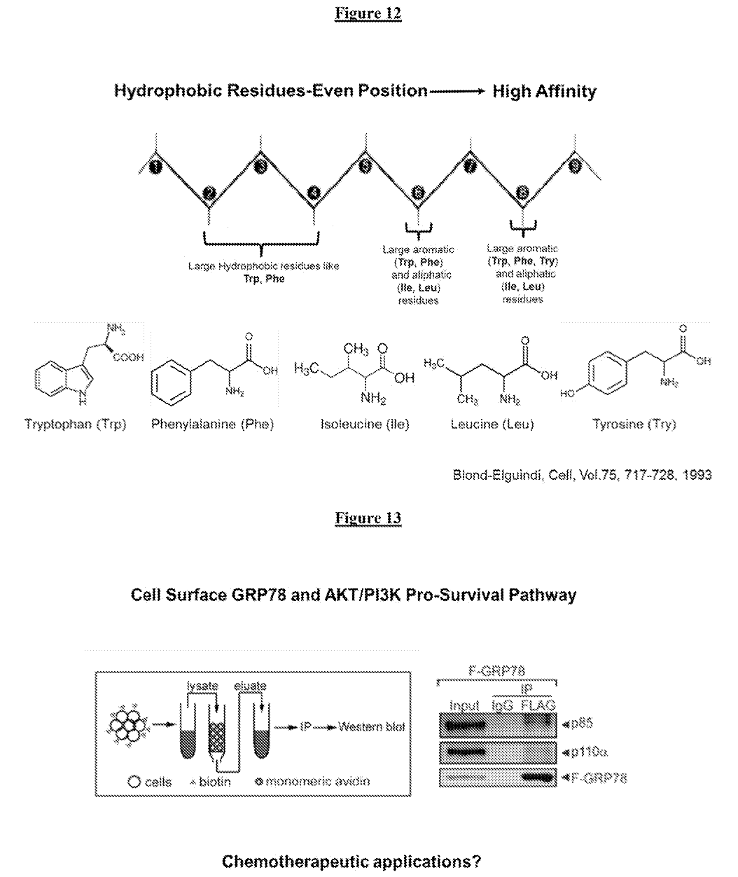

SK-2 is a 8 amino acid linear peptide sequence that is designed based on a scoring system which is obtained by a phage display study (Sylvie Blond-Elguindi et. al. Cell (1993), Vol. 75, 717-728) (FIG. 2A; see also FIG. 10). The SK-2 peptide has an amino acid sequence: WIFPWIQL (SEQ ID NO:3).

N-DOTA-SK2.

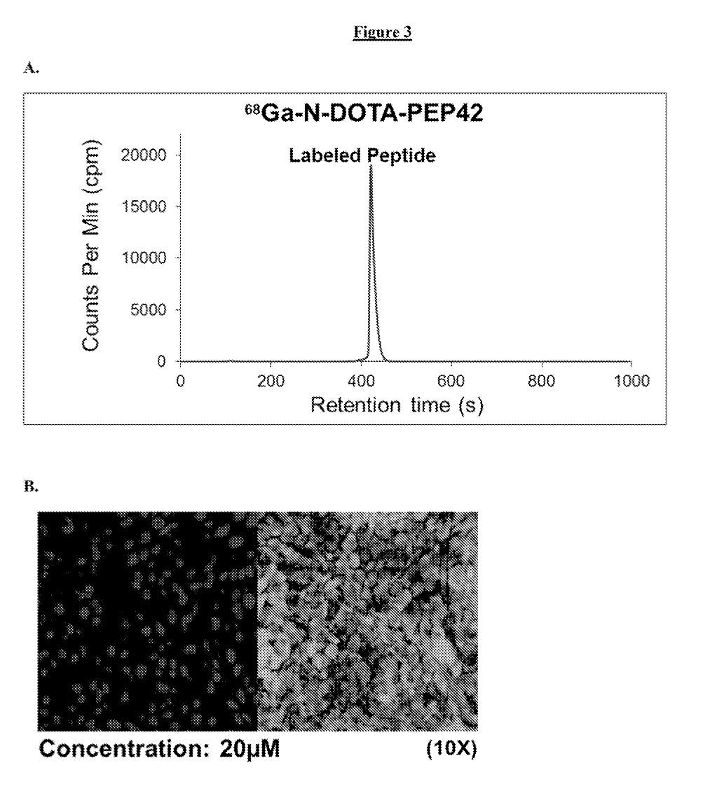

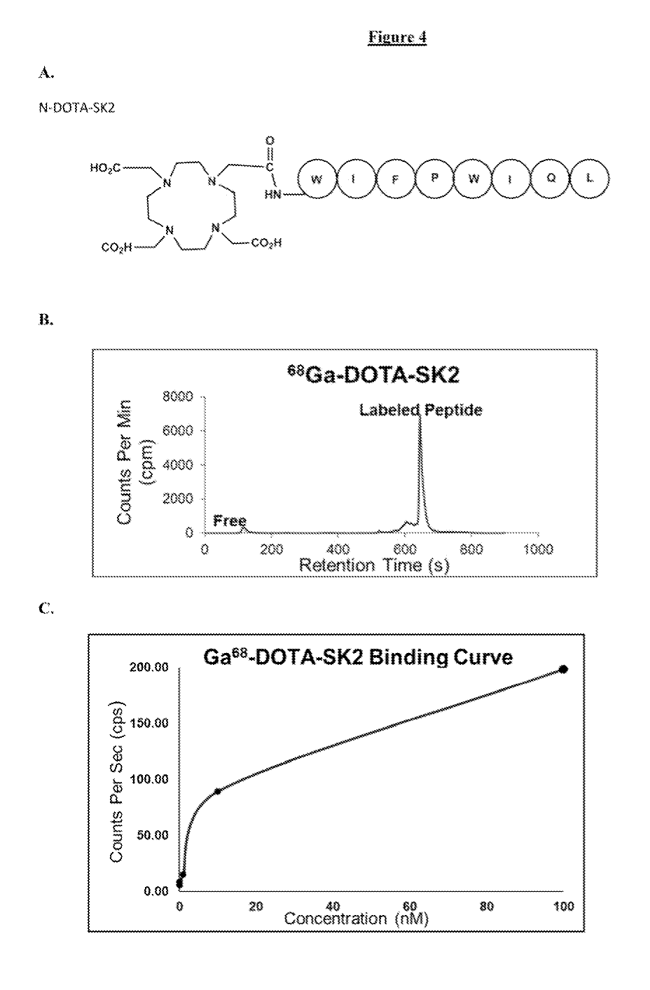

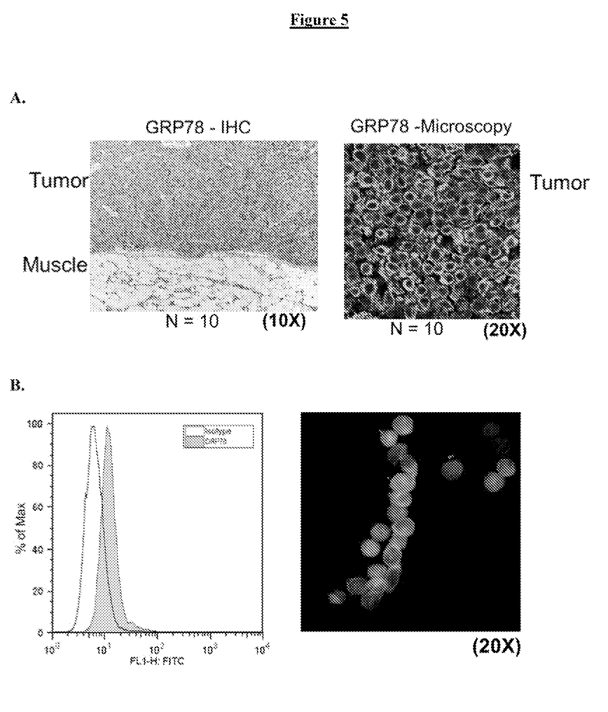

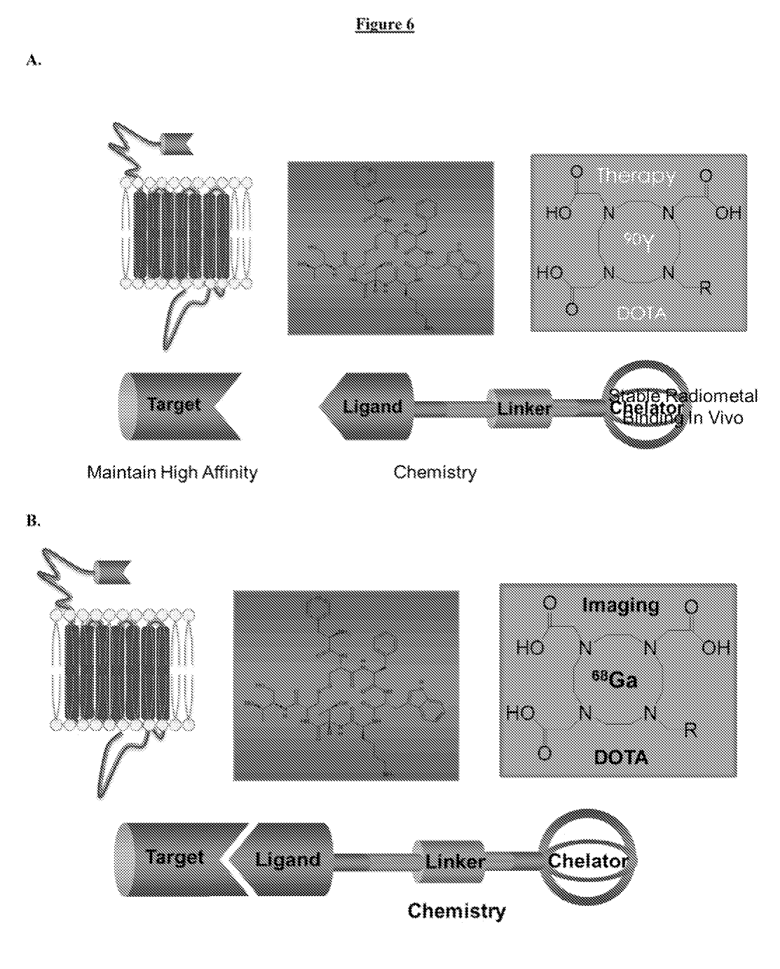

DOTA was conjugated to the N-terminus of the peptide (FIGS. 2B and 4A). It was labeled with ⁶⁸Ga with a high efficiency (Specific activity: 1.37 MBq/nmole) (see, FIG. 4B). 1-LN (10⁶) cells were incubated with increasing concentration of ⁶⁸Ga labeled peptide (0.01-100 nM) for an hour to obtain a saturation curve. The unbound peptide was removed by washing once with PBS. The counts per sec (cps) was obtained on a gamma counter (see, FIG. 4C).

### GRP78 Expression

Variation in GRP78 expression was studied in B16 melanoma tumors, HepG2 and melanocytes by immunochemistry (IHC).

### Results

IHC staining of melanoma tumor cross sections shows a remarkable difference in the expression of GRP78 in tumors vs adjacent tissue (FIG. 5A). Immunofluorescence studies show expression of GRP78 on the surface of HepG2 cells increases upon incubation with 50 μM hydrogen peroxide—evidence that enhanced expression can be induced by oxidative stress. Fluorescein-labeled-cyclized derivatives were internalized and binding affinity did not depend on the position of DOTA/fluorescein. DOTA derivatives were efficiently labeled with Ga-68. Additional experiments may be performed to evaluate the effect of hypoxia, redox imbalance and ER stress induces on the cell surface expression of GRP78.

### Conclusion

As described herein, GRP78 is a potential target for PET imaging and targeted therapy for cancer. Cell surface expression appears to be mediated by stress mechanisms. DOTA conjugated GRP78 targeted peptides can be labeled with Ga-68 for PET. It has been hypothesized that the peptides with large hydrophobic amino acid residues at even positions bind to GRP78 with a higher affinity.

### Example 2

### Cell-Surface Expression of GRP78 in Melanoma.

Emerging evidence suggests that glucose regulated protein (GRP78) has potential as a new cell-surface target for metastatic melanoma. GRP78 has long been known as a molecular chaperone and master regulator of the unfolded protein response (UPR) confined to the endoplasmic reticulum (ER). However, a growing body of evidence demonstrates that tumor-cell stresses not only increase intracellular GRP78, but also lead to elevated cell-surface GRP78 in melanoma cells. In addition, clinical evidence correlates cell-surface GRP78 with melanoma progression, suggesting a protective role in melanoma.

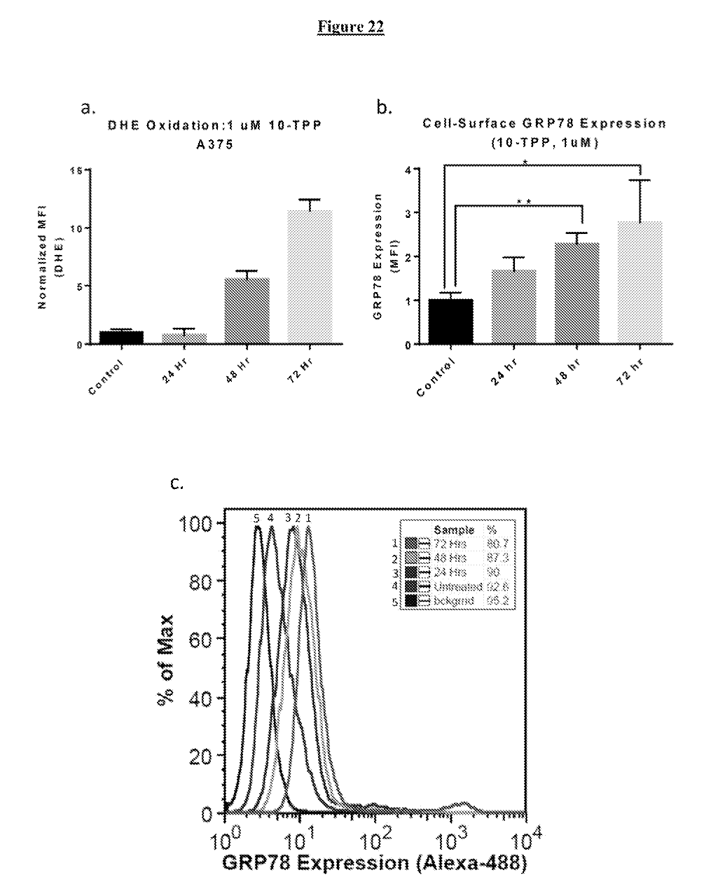

The expression of GRP78 was examined in B-16 mouse melanoma tissue using immunohistochemistry (IHC) and immunofluorescence (FIG. 21). The IHC revealed significantly higher GRP78 expression in the tumor tissue relative to the surrounding muscle tissue. Additionally, increases in DHE oxidation shows increasing levels of ROS, which lead to increases in cell-surface GRP78 expression (FIG. 22).

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A conjugate of formula (I):
P-(X-D)ₙ (I)
wherein:
P is a peptide that binds to a glucose regulated protein 78 (GRP78);
X is a direct bond or a linking group;
D is a detectable agent; and
n is 1 to 4.

2-4. (canceled)

5. The conjugate of claim 1, wherein the peptide comprises at least one hydrophobic amino acid selected from leucine, isoleucine, phenylalanine, tryptophan, valine, tyrosine, alanine and cysteine, and wherein the at least one hydrophobic amino acid is located at an even position within the peptide.

6-9. (canceled)

10. The conjugate of claim 1, wherein the peptide comprises an amino acid sequence selected from formulas (II), (III), (IV), (V) and (VI):
X₁-A₁-A₁-A₂-A₂-A₂-A₃-A₃-A₃-A₄-A₂-A₅-A₅-A₁-X₁ (II)
X₁-A₀-A₇-A₀-A₂-A₀-A₁-A₀-A₅-A₀-A₃-A₀-A₃-X₁ (III)
X₁-A₀-A₂-A₀-A₃-A₀-A₂-A₀-A₂-X₁ (IV)
X₁-A₀-A₄-A₀-A₄-A₀-A₂-A₀-A₂-X₁ (V)
X₁-A₀-A₄-A₀-A₄-A₀-A₆-A₀-A₆-X₁ (VI)
wherein:
X₁ is 0-8 amino acids;
A₀ is any amino acid;
A₁ is independently an amino acid with a polar neutral side chain;
A₂ is independently an amino acid with a hydrophobic aliphatic side chain;
A₃ is independently a unique amino acid;
A₄ is independently an amino acid with a hydrophobic aromatic side chain;
A₅ is independently an amino acid with a basic side chain;
A₆ is independently an amino acid with a hydrophobic aliphatic or aromatic side chain; and
A₇ is independently an amino acid with an acidic side chain.

11. The conjugate of claim 10, wherein:
A₁ is independently selected from asparagine, cysteine, glutamine, methionine, serine and threonine;
A₂ is independently selected from alanine, isoleucine, leucine and valine;
A₃ independently selected from glycine and proline;
A₄ is independently selected from phenylalanine, tryptophan and tyrosine;
A₅ is independently selected from arginine, histidine and lysine;
A₆ is independently selected from alanine, isoleucine, leucine, valine, phenylalanine, tryptophan and tyrosine; and/or
A₇ is independently selected from aspartic acid and glutamic acid.

12-18. (canceled)

19. The conjugate of claim 10, wherein the peptide comprises an amino acid sequence selected from the group consisting of: CTVALPGGYVRKC (SEQ ID NO:1), WDLAWMFRLPVG (SEQ ID NO:2), WIFPWIQL (SEQ ID NO:3), GWAFSIPL (SEQ ID NO:4).

20. (canceled)

21. The conjugate of claim 10, wherein the peptide comprises an amino acid sequence selected from formulas (IIIa), (IVa), (Va) and (VIa):
X₁-A₄-A₇-A₂-A₂-A₄-A₁-A₄-A₅-A₂-A₃-A₂-A₃-X₁ (IIIa)
X₁-A₄-A₂-A₄-A₃-A₄-A₂-A₁-A₂-X₁ (IVa)
X₁-A₃-A₄-A₂-A₄-A₁-A₂-A₃-A₂-X₁ (Va)
X₁-A₀-A₄ₐ-A₀-A₄ₐ-A₀-A₆ₐ-A₀-A₆ₐ-X₁ (VIa)
wherein:
A₄ₐ is independently selected from tryptophan and phenylalanine;
A₆ₐ is independently selected from tryptophan, phenylalanine, isoleucine, leucine and tyrosine.

22-35. (canceled)

36. The conjugate of claim 1, wherein X is a divalent, branched or unbranched, saturated or unsaturated, hydrocarbon chain, having from 2 to 25 carbon atoms, wherein one or more (e.g. 1, 2, 3, or 4) of the carbon atoms is optionally replaced by (—O—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

37. (canceled)

38. The conjugate of claim 1, wherein D comprises a chelating group, wherein the chelating group optionally includes a radionuclide.

39. The conjugate of claim 38, wherein the chelating group is selected from

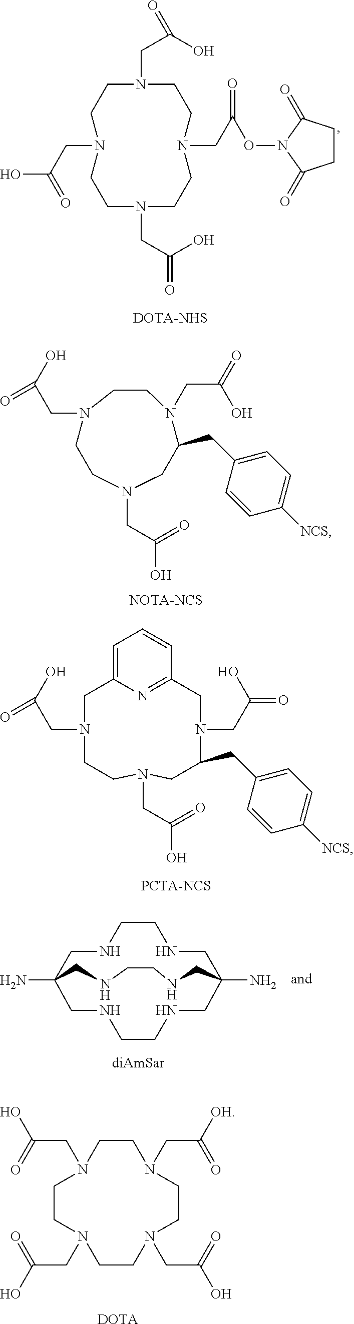

40-42. (canceled)

43. The conjugate of claim 38, wherein the radionuclide is selected from Antimony-124, Antimony-125, Arsenic-74, Barium-103, Barium-140, Beryllium-7, Bismuth-206, Bismuth-207, Cadmium-109, Cadmium-115m, Calcium-45, Cerium-139, Cerium-141, Cerium-144, Cesium-137, Chromium-51, Cobalt-55, Cobalt-56, Cobalt-57, Cobalt-58, Cobalt-60, Cobalt-64, Copper-64, Copper-67, Erbium-169, Europium-152, Gallium-64, Gallium-68, Gadolinium-153, Gadolinium-157 Gold-195, Gold-199, Hafnium-175, Hafnium-175-181, Holmium-166, Indium-110, Indium-111, Iridium-192, Iron-55, Iron-59, Krypton-85, Lead-210, Manganese-54, Mercury-197, Mercury-203, Molybdenum-99, Neodymium-147, Neptunium-237, Nickel-63, Niobium-95, Osmium-185+191, Palladium-103, Platinum-195m, Praseodymium-143, Promethium-147, Protactinium-233, Radium-226, Rhenium-186, Rhenium-188, Rubidium-86, Ruthenium-103, Ruthenium-106, Scandium-44, Scandium-46, Selenium-75, Silver-110m, Silver-111, Sodium-22, Strontium-85, Strontium-89, Strontium-90, Sulfur-35, Tantalum-182, Technetium-99m, Tellurium-125, Tellurium-132, Thallium-204, Thorium-228, Thorium-232, Thallium-170, Tin-113, Tin-114, Tin-117m, Titanium-44, Tungsten-185, Vanadium-48, Vanadium-49, Ytterbium-169, Yttrium-86, Yttrium-88, Yttrium-90, Yttrium-91, Zinc-65, and Zirconium-95.

44. (canceled)

45. The conjugate of claim 1, wherein D comprises a fluorescent group.

46-47. (canceled)

48. The conjugate of claim 1, wherein a conjugate of formula (I) is selected from:

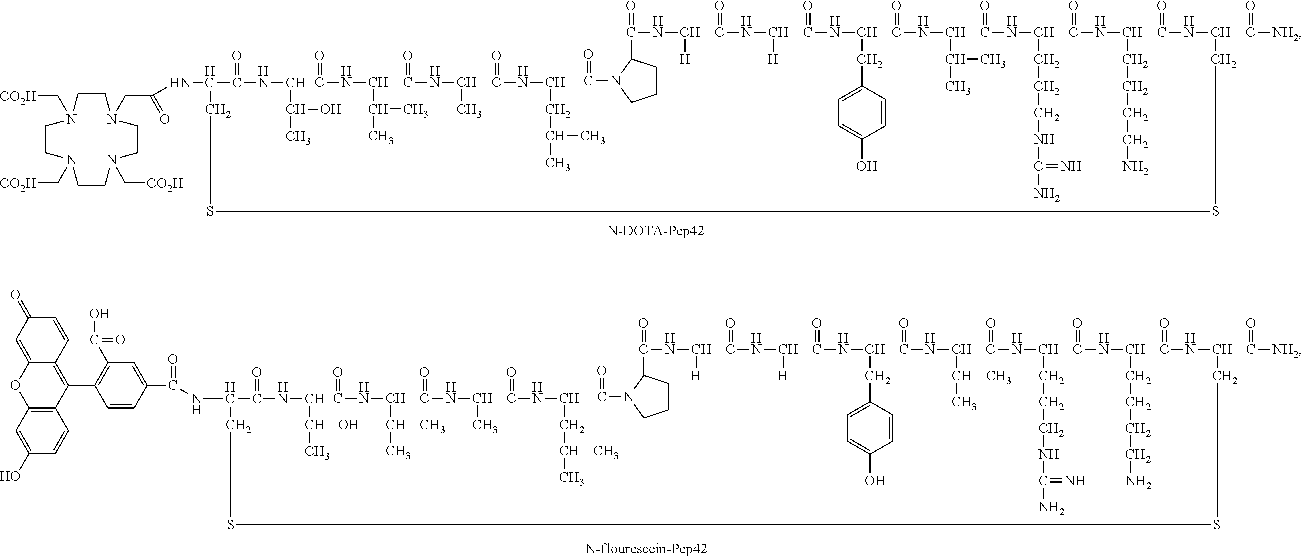

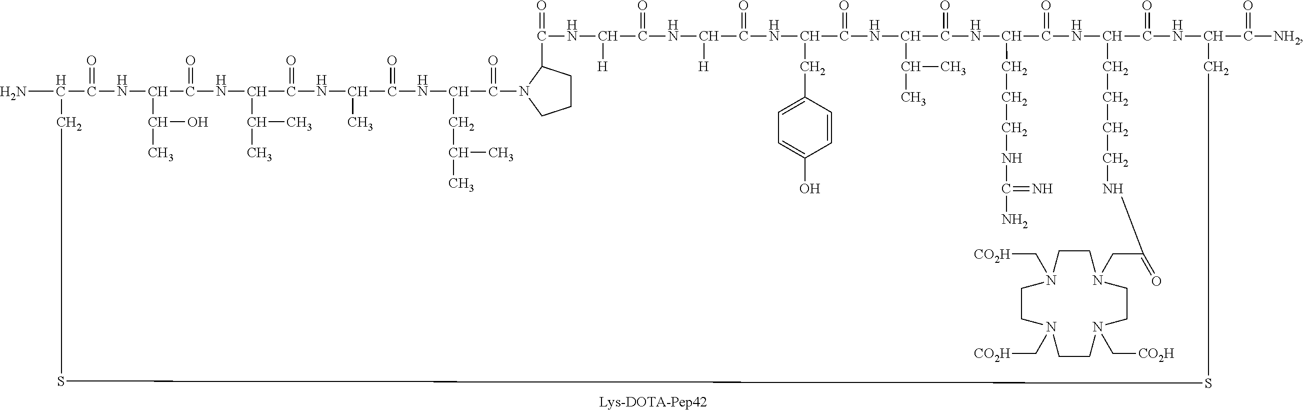 [SMILES: CC(C)CC1NC(=O)C(C)NC(=O)C(C(C)C)NC(=O)C(C(C)O)NC(=O)C(N)CSSCC(C(N)=O)NC(=O)C(CCCCNC(=O)CN2CCN(CC(=O)O)CCN(CC(=O)O)CCN(CC(=O)O)CC2)NC(=O)C(CCCNC(=N)N)NC(=O)C(C(C)C)NC(=O)C(Cc2ccc(O)cc2)NC(=O)CNC(=O)CNC(=O)C2CCCN2C1=O]

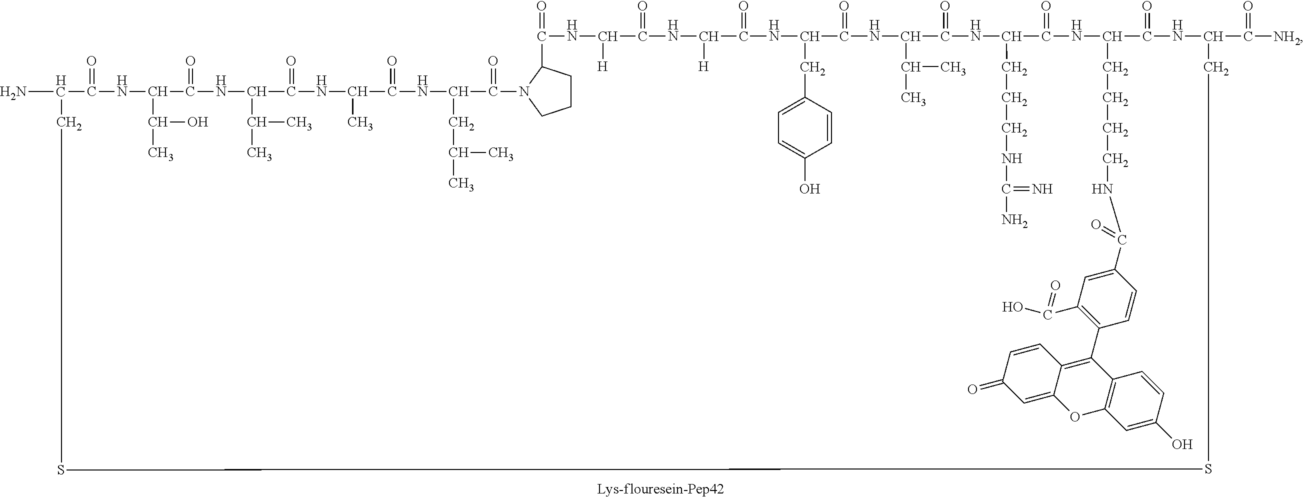 [SMILES: CC(C)CC1NC(=O)C(C)NC(=O)C(C(C)C)NC(=O)C(C(C)O)NC(=O)C(N)CSSCC(C(N)=O)NC(=O)C(CCCCNC(=O)c2ccc(-c3c4ccc(=O)cc-4oc4cc(O)ccc34)c(C(=O)O)c2)NC(=O)C(CCCNC(=N)N)NC(=O)C(C(C)C)NC(=O)C(Cc2ccc(O)cc2)NC(=O)CNC(=O)CNC(=O)C2CCCN2C1=O]

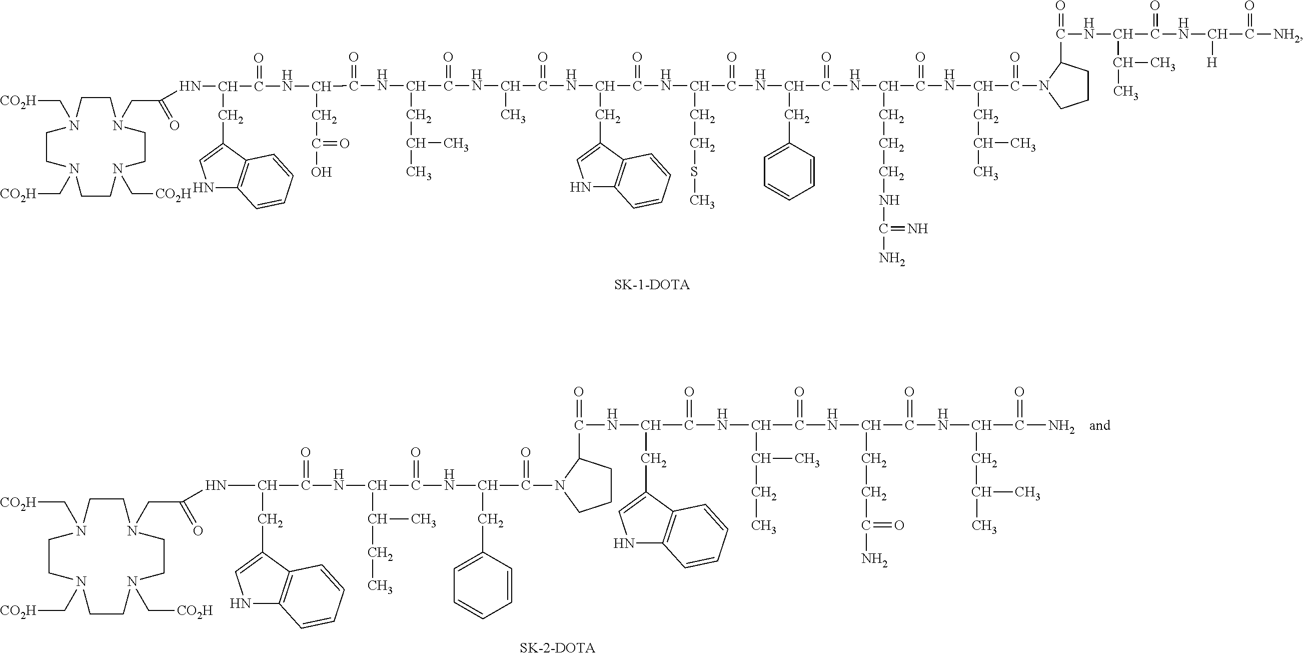 [SMILES: CSCCC(NC(=O)C(Cc1c[nH]c2ccccc12)NC(=O)C(C)NC(=O)C(CC(C)C)NC(=O)C(CC(=O)O)NC(=O)C(Cc1c[nH]c2ccccc12)NC(=O)CN1CCN(CC(=O)O)CCN(CC(=O)O)CCN(CC(=O)O)CC1)C(=O)NC(Cc1ccccc1)C(=O)NC(CCCNC(=N)N)C(=O)NC(CC(C)C)C(=O)N1CCCC1C(=O)NC(C(=O)NCC(N)=O)C(C)C ; CCC(C)C(NC(=O)C(Cc1c[nH]c2ccccc12)NC(=O)C1CCCN1C(=O)C(Cc1ccccc1)NC(=O)C(NC(=O)C(Cc1c[nH]c2ccccc12)NC(=O)CN1CCN(CC(=O)O)CCN(CC(=O)O)CCN(CC(=O)O)CC1)C(C)CC)C(=O)NC(CCC(N)=O)C(=O)NC(CC(C)C)C(N)=O ; "and"]

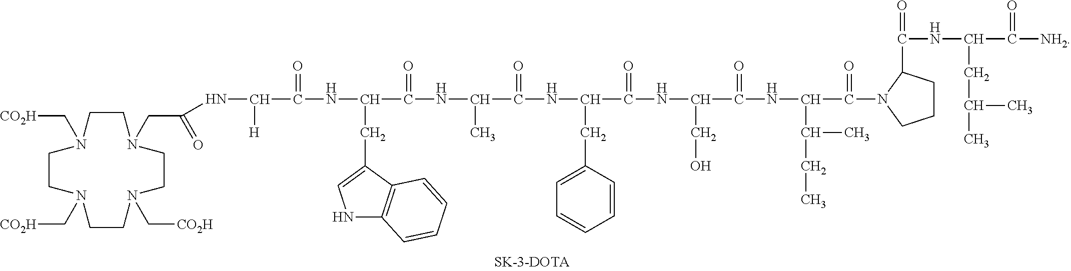 [SMILES: CCC(C)C(NC(=O)C(CO)NC(=O)C(Cc1ccccc1)NC(=O)C(C)NC(=O)C(Cc1c[nH]c2ccccc12)NC(=O)CNC(=O)CN1CCN(CC(=O)O)CCN(CC(=O)O)CCN(CC(=O)O)CC1)C(=O)N1CCCC1C(=O)NC(CC(C)C)C(N)=O]

49. A GRP78 targeting peptide comprising amino acid sequence GWAFSIPL (SEQ ID NO:4).

50. A pharmaceutical composition comprising the conjugate of claim 1 and a pharmaceutically acceptable excipient.

51. A method for treating or preventing cancer in an animal comprising administering a therapeutically effective amount of a conjugate of claim 1 to the animal.

52-55. (canceled)

56. A method of detecting a GRP78 molecule, comprising contacting a cell with a conjugate of claim 1.

57. A method of detecting cancer cells in a test tissue sample, comprising contacting the test sample with a conjugate of claim 1 and measuring a signal from the detectable agent, wherein a signal from the test sample that is greater than a signal from a non-cancerous control sample indicates the presence of cancer cells in the test tissue sample.

58. (canceled)

59. A method of detecting cancer in an animal, comprising administering a conjugate of claim 1 to the animal and measuring a signal from the detectable agent, wherein a signal greater than a signal from a control animal without cancer indicates the animal has cancer.

60-61. (canceled)

62. A method of determining the effectiveness of a cancer therapy in an animal, comprising
1) administering a conjugate of claim 1 to the animal and measuring a first signal from the detectable agent;
2) administering a cancer therapy;
3) administering a conjugate of claim 1 to the animal and measuring a second signal from the detectable agent; and
4) comparing the first signal with the second signal, wherein the cancer therapy is effective if the second signal is less than the first signal.

63-64. (canceled)

65. A kit comprising:
1) a conjugate of claim 1;
2) instructions for loading a radionuclide into the conjugate to generate a radiolabeled conjugate;
4) instructions for administering the radiolabeled conjugate to an animal; and
5) optionally, a radionuclide.

66. (canceled)


SEQUENCE LISTING (4 sequences)

SEQ ID NO: 1 - protein, 13 aa, Artificial Sequence
  Description of Artificial Sequence Synthetic peptide
CTVALPGGYVRKC

SEQ ID NO: 2 - protein, 12 aa, Artificial Sequence
  Description of Artificial Sequence Synthetic peptide
WDLAWMFRLPVG

SEQ ID NO: 3 - protein, 8 aa, Artificial Sequence
  Description of Artificial Sequence Synthetic peptide
WIFPWIQL

SEQ ID NO: 4 - protein, 8 aa, Artificial Sequence
  Description of Artificial Sequence Synthetic peptide
GWAFSIPL